(19) United States
(12) Patent Application Publication
(10) Pub. No.: US 2009/0148887 A1
(43) Pub. Date: Jun. 11, 2009

(54) GENETICALLY ENCODED BORONATE AMINO ACID

(75) Inventors: ERIC BRUSTAD, Sierra Madre, CA (US); MARK L. BUSHEY, NEW YORK, NY (US); PETER G. SCHULTZ, LA JOLLA, CA (US)

Correspondence Address: QUINE INTELLECTUAL PROPERTY LAW GROUP, P.C., P O BOX 458, ALAMEDA, CA, 94501, US

(73) Assignee: THE SCRIPPS RESEARCH INSTITUTE, LA JOLLA, CA (US)

(21) Appl. No.: 12/262,025

(22) Filed: Oct. 30, 2008

Related U.S. Application Data
(60) Provisional application No. 61/001,681, filed on Nov. 2, 2007.
(60) Provisional application No. 61/127,262, filed on May 8, 2008.
(60) Provisional application No. 61/137,689, filed on Aug. 1, 2008.
(60) Provisional application No. 61/189,739, filed on Aug. 22, 2008.
(60) Provisional application No. 61/194,773, filed on Sep. 29, 2008.

(51) Int. Cl. C12P 21/00; C12N 9/00; C12N 5/06; C12N 1/21; C12N 1/19

(52) U.S. Cl. 435/68.1; 435/183; 435/325; 435/252.33; 435/254.2

(57) ABSTRACT

Provided are compositions comprising an aminoacyl tRNA synthetase that selectively recognizes a boronic amino acid. Methods of incorporating a boronic amino acid into a target polypeptides and target polypeptides produced by the methods are also provided. Methods of producing a protein, which methods comprise site-specifically encoding a boronic amino acid residue into a mutant protein and selectively converting the boronic amino acid residue into a natural amino acid residue are provided. Also provided are compositions comprising a solid phase matrix covalently bound to a polypeptide through a boronic amino acid residue. In addition, compositions comprising a purified population of polypeptide molecules that each comprise a borono amino acid at a selected site are provided.

### CROSS-REFERENCE TO RELATED APPLICATIONS

This application claims priority to and benefit of U.S. Provisional Patent Application Ser. No. 61/001,681, entitled, “Directed evolution using proteins comprising unnatural amino acids,” by Liu, et al., filed on Nov. 2, 2007; Provisional Patent Application Ser. No. 61/127,262, entitled, “Directed evolution using proteins comprising unnatural amino acids,” by Liu, et al., filed on May 8, 2008; U.S. Provisional Patent Application Ser. No. 61/137,689, entitled, “A genetically encoded boronate amino acid,” by Brustad, et al., filed on Aug. 1, 2008; U.S. Provisional Patent Application Ser. No. 61/189,739, entitled, “A genetically encoded boronate amino acid,” by Brustad, et al., filed on Aug. 22, 2008; and U.S. Provisional Patent Application Ser. No. 61/194,773, entitled, “Directed evolution using proteins comprising unnatural amino acids,” by Liu, et al., filed on Sep. 29, 2008; the contents of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support from the National Institutes of Health under Grant No. 5R01 GM62159. The government has certain rights to this invention.

### FIELD OF THE INVENTION

The invention is in the field of translation biochemistry. The invention provides compositions and methods for using orthogonal tRNAs, orthogonal aminoacyl-tRNA synthetases, and pairs thereof, that incorporate boronic amino acids into proteins. The invention also relates to methods of producing target proteins in cells using such pairs, target proteins made by the methods, and uses for such target proteins.

### BACKGROUND OF THE INVENTION

Organoborates have attracted considerable interest as synthetic intermediates in a variety of contexts. These include Suzuki cross-coupling reactions (Miyaura and Suzuki (1995) “Palladium-Catalyzed Cross-Coupling Reactions of Organoboron Compounds,” Chemical Reviews 95: 2457; Suzuki (1999) “Recent advances in the cross-coupling reactions of organoboron derivatives with organic electrophiles, 1995-1998,” Journal of Organometallic Chemistry 576:147), copper catalyzed heteroatom alkylation reactions (Chan, et al. (2003) “Copper promoted C—-N and C—-O bond cross-coupling with phenyl and pyridylboronates,” Tetrahedron Letters 44:3863), asymmetric reductions (Huang, et al (2000) “Asymmetric reduction of acetophenone with borane catalyzed by chiral oxazaborolidinone derived from L-a-amino acids,” Synthetic Communications 30:2423), Diels-Alder reactions (Ishihara and Yamamoto (1999) “Arylboron Compounds as Acid Catalysts in Organic Synthetic Transformations,” European Journal of Organic Chemistry 527), as well as a variety of other transformations.

Boronic acids are also known to form reversible covalent complexes with diols (Lorand and Edwards, (1959) “Polyol Complexes and Structure of the Benzeneboronate Ion,” Journal of Organic Chemistry 24:769), amino alcohols (Springsteen, et al. (2001) “The Development of Photometric Sensors for Boronic Acids,” Bioorganic Chemistry 29:259), amino acids (Mohler and Czarnik (1994) “Amino acid Chelative complexation by an Arylboronic Acid,” Journal of the American Chemical Society 116:2233; Mohler and Czarnik (1993) “Alpha-Amino-Acid Chelative Complexation by an Arylboronic Acid,” Journal of the American Chemical Society 115: 7037) alkoxides (Cammidge and Crépy (2004) “Synthesis of chiral binaphthalenes using the asymmetric Suzuki reaction,” Tetrahedron 60:4377), and hydroxamic acids (Lamandé, et al. (1980) “Structure et acidite de composes a atome de bore et de phosphore hypercoordonnes,” Journal of Organometallic Chemistry 329).

This latter property has been exploited in the synthesis of ligands for the selective recognition of sugars (James, et al. (1996) “Saccharide Sensing with Molecular Receptors Based on Boronic Acid,” Angewandte Chemie-International Edition in English 35:1910; James, et al. (1995) “Chiral discrimination of monosaccharides using a fluorescent molecular sensor,” Nature 374:345; Wang, et al. (2002) “Boronic Acid-Based Sensors,” Current Organic Chemistry 6:1285) and for the development of potent serine protease inhibitors (Adams, et al. (1998) “Potent and selective inhibitors of the proteasome: Dipeptyidyl boronic acids,” Bioorganic & Medicinal Chemistry Letters 8:333; Weston, et al. (1998) “Structure-Based Enhancement of Boronic Acid Inhibitors of AmpC b-Lactamase,” Journal of Medicinal Chemistry 41:4577; Yang, et al. (2003) “Boronic acid compounds as potential pharmaceutical agents,” Medicinal Research Reviews 23:346; Matthews, et al. (1975) “X-ray crystallographic study of boronic acid adducts with subtilisin BPN′ (Novo). A model for the catalytic transition state,” Journal of Biological Chemistry 250:7120). In addition, boronates are finding utility as boron neutron capture agents to kill tumor cells (Kinashi, et al. (2002) “Mutagenic effect of borocaptate sodium and boronophenylalanine in neutron capture therapy,” International Journal of Radiation Oncology Biology Physics 54:562).

Despite their unique and highly useful chemical properties, boronic acids are not known to occur naturally in polypeptides, either as posttranslational modifications or as cofactors. The ability to genetically encode boronic amino acids could, potentially, provide highly useful tools for protein purification, biomolecular recognition, selective chemical modification, and even therapeutic use of a variety of proteins. The present invention provides for these and other features that will be apparent upon review of the following.

### SUMMARY OF THE INVENTION

The invention is generally directed to methods and compositions for the incorporation of boronic amino acids, e.g., an aliphatic, aryl or heterocycle substituted boronic acid, a p-boronophenylalanine, an o-boronophenylalanine, or an m-boronophenylalanine, into target polypeptides response to a selector codon. These compositions include orthogonal aminoacyl tRNA synthetases (O-RSs) that do not substantially interact with or interfere with the endogenous components of the translation system in which they are being used. The chemical properties of boronic amino acids allow the polypeptide into which they have been incorporated to be used as a substrate in one or more of a variety of reactions, e.g., a labeling reaction, a substrate for probe addition, a substrate for an oxidation reaction, a substrate for a reduction reaction, a substrate for an esterification reaction, a substrate for a saccharide addition reaction, a substrate for a PEG addition reaction, a substrate for a Suzuki cross-coupling reaction, a substrate for a transition metal catalyzed reaction, a substrate for a palladium catalyzed reaction, a substrate for a copper catalyzed heteroatom alkylation reaction, a substrate for an asymmetric reduction, a substrate for a Diels-Alder reaction, or the like.

In addition to the many uses for a new protein reactive group in labeling, protein engineering, protein stability, chemical modification, and the like, the methods and compositions provided by the invention are also particularly useful in therapeutic applications. Boronic amino acid labeled proteins can be used, e.g., to selectively kill target cells, e.g., as a treatment against infectious agents, to treat cancer by killing tumor cells, or to treat other diseases where death of the target cell is desirable.

Thus, in a first aspect, the invention provides compositions that comprise an aminoacyl tRNA synthetase that selectively recognizes a boronic amino acid, e.g., an aliphatic, aryl or heterocycle substituted boronic acid, a p-boronophenylalanine, an o-boronophenylalanine, an m-boronophenylalanine, or the like. In this context, “selective recognition” indicates that the synthetase charges a cognate O-tRNA with the boronic amino acid more efficiently than with any natural amino acid. For example, the ORS may have one or more of: a higher k_{cat}, or a lower Kₘ for the boronic amino acid than for any natural amino acid. The synthetase of the compositions can optionally be homologous to a wild-type tyrosyl tRNA synthetase from Methanococcus jannaschi. In certain embodiments of the compositions, the synthetase optionally comprises a Ser or Gly residue at position 32, an alanine at position 65, a His or Met residue at position 70, a Ser or Ala residue at position 158, a glutamine at position 162 or a combination thereof, wherein amino acid position numbering corresponds to amino acid position numbering of the wild-type tyrosyl tRNA synthetase. Optionally, the synthetase can comprise or be encoded by: 1BF6, 1BF9, 1BE3, 1BF10, 1BF12, 1BG10, or 1BG11.

The compositions of the invention can optionally comprise a cell, e.g., a prokaryotic, e.g., bacterial cell, e.g., an E. coli cell, or a eukaryotic cell (plant cell, animal cell, yeast cell, mammal cell, etc.) in which the aminoacyl tRNA synthetase is expressed. In such embodiments, the expressed synthetase is orthogonal to the cell, and the cell further expresses a cognate orthogonal tRNA (O-tRNA) that is selectively charged by the synthetase with the boronic amino acid. For example, the O-tRNA expressed by the cell can optionally comprise a tRNA from the sequence listing. The cell can optionally encode a target nucleic acid that encodes a selector codon, e.g., a stop codon, a rare codon, a nonsense codon, or a 4- or more base codon, that is selectively recognized by the O-tRNA, such that a boronic amino acid residue can be specifically incorporated into a target polypeptide in the cell in response to the selector codon.

The target polypeptide comprising the boronic amino acid residue can optionally be a substrate for a labeling reaction, a substrate for probe addition, a substrate for an oxidation reaction, a substrate for a reduction reaction, a substrate for an esterification reaction, a substrate for a polyol addition reaction, a substrate for a saccharide addition reaction, a substrate for a PEG addition reaction, a substrate for a Suzuki cross-coupling reaction, a substrate for a transition metal catalyzed reaction, a substrate for a palladium catalyzed reaction, a substrate for a copper catalyzed heteroatom alkylation reaction, a substrate for an asymmetric reduction, or a substrate for a Diels-Alder reaction, wherein the respective reaction selectively acts on the boronic amino acid residue. The target polypeptide can optionally be, for example, a therapeutic protein, a cytokine, a growth factor, an immunogen, an enzyme, a cell receptor ligand, a modulator of a serine protease, an inhibitor of a serine protease, a modulator of a glycosylated macromolecule, an inhibitor of a glycosylated macromolecule, a saccharide binding protein, an oligosaccharide binding protein, an antibody, an antibody fragment, a therapeutic antibody, an antibody or antibody fragment that specifically binds to a glycoprotein, an antibody that specifically binds to a serine protease, an antibody that specifically binds to a serum protease, a phage display protein, or a cancer cell ligand.

In a related aspect, the invention provides methods of incorporating a boronic amino acid, e.g., an aliphatic, aryl or heterocycle substituted boronic acid, a p-boronoamino acid, an o-boronophenylalanine, or an m-boronophenylalanine, into a target polypeptide. These methods include, e.g., providing a translation system that includes an orthogonal aminoacyl tRNA synthetase (O-RS) selective for the boronic amino acid, a cognate orthogonal tRNA (O-tRNA) specific for a selector codon, and a target nucleic acid comprising the selector codon that encodes the target polypeptide. The methods also typically include permitting the translation system to incorporate a boronic amino acid residue into the target polypeptide during translation of the target nucleic acid into the target polypeptide. The invention also provides the target polypeptides produced by these methods.

The O-RS used in the methods can optionally be homologous to a wild-type tyrosyl tRNA synthetase from Methanococcus jannaschii which comprises a Ser or Gly residue at position 32, an alanine at position 65, a His or Met residue at position 70, a Ser or Ala residue at position 158, a glutamine at position 162, or a combination thereof, with the position numbering corresponding to positions of the wild-type tyrosyl tRNA synthetase.

The methods can optionally include forming a covalent bond between the boronic acid and an additional residue (e.g., serine, tyrosine, or threonine) of the target polypeptide, thereby stabilizing the polypeptide. Optionally, the methods can separately or additionally include forming a covalent bond between the boronic amino acid and a residue of an additional target polypeptide, e.g., a serine protease, a serum protease, an antibody or antibody ligand, or a glycosylated polypeptide; or macromolecule, e.g., a macromolecule comprising a saccharide or an oligosaccharide, e.g., a glycosylated macromolecule. Optionally, the target polypeptide can be an antibody or fragment thereof, and the additional target polypeptide can optionally comprise an epitope recognized by the antibody or fragment.

The target polypeptide produced by the methods can optionally comprise a ligand that is selectively bound or internalized by a target cell. The target cell can optionally be a cell targeted for destruction, such as a tumor cell, an infectious cell, or the like. The methods can optionally include contacting the tumor or other target cell with the target polypeptide, which can result in target cell death. Optionally, the tumor or other target cell can be present in an organism, and contacting the tumor cell with the target polypeptide can optionally comprise local or systemic delivery of the target polypeptide to the organism. The method can also optionally further comprise irradiating the target cell, e.g., with neutrons, e.g., producing a localized field of, e.g., α particles which damage or kill the tumor cell.

The methods provided by the invention can further include performing a labeling reaction, a probe addition reaction, an oxidation reaction, a reduction reaction, an esterification reaction, a polyol addition reaction, a saccharide addition reaction, a PEG addition reaction, a Suzuki cross-coupling reaction, a transition metal catalyzed reaction, a palladium catalyzed reaction, a copper catalyzed heteroatom alkylation reaction, an asymmetric reduction, or a Diels-Alder reaction on the target polypeptide. The esterification reaction can optionally comprise esterification of the boronic residue with an alcohol, a diol, a polyol, a saccharide, an amino alcohol, a PEG, a diamine compound, or the like. The labeling reaction optionally includes incubation of the target polypeptide with a conjugate of interest, such as a biophysical reporter moiety, an active group, a protective group, or the like, or incubation of the target polypeptide with a label moiety, e.g., a moiety comprising a polyhydroxylated moiety, e.g., sorbitol or a glucamine moiety. Optionally, the label moiety can comprise a fluorescent or luminescent moiety. In one example, the Suzuki cross-coupling reaction can optionally be performed using a palladium catalyst and/or can optionally result in the covalent attachment of an aryl iodide to the target polypeptide.

The methods can optionally further include affinity purification of the target polypeptide by binding the target polypeptide to a purification matrix that comprises a moiety that binds to the boronic amino acid residue. The purification matrix can optionally comprise a polyhydroxylated moiety, a saccharide or a polysaccharide. For example, the matrix can comprise n-methylglucamine. Affinity purification of the target polypeptide can optionally include selectively oxidizing the boronic amino acid residue, e.g., a p-boronophenylalanine residue, to produce a natural amino acid residue, e.g., a tyrosine residue, or selectively reducing said boronic amino acid residue to produce a phenylalanine residue. This approach, termed “scarless purification,” has the high yield single-step purification advantages of affinity tag purification, while providing an eventual protein product that lacks the affinity tag. This is an advantage, because an affinity tag can have undesirable properties for a purification product.

In another aspect, the invention provides methods of producing a protein that include site-specifically encoding a boronic amino acid residue, e.g., a p-boronophenylalanine, into a mutant protein and selectively converting the boronic amino acid residue into a natural amino acid residue, e.g., a tyrosine or phenylalanine residue, thereby producing the protein. The methods can optionally include purifying the mutant protein by binding the boronic amino acid residue to a purification matrix that binds to the residue prior to converting the boronic amino acid residue into the natural amino acid residue.

The boronic amino acid residue can optionally be incorporated into the mutant protein in a cell that comprises an orthogonal aminoacyl tRNA synthetase (O-RS) that is selective for the boronic amino acid. The O-RS used in these methods can optionally be homologous to a wild-type tyrosyl tRNA synthetase from Methanococcus jannaschii which comprises a Ser or Gly residue at position 32, an alanine at position 65, a His or Met residue at position 70, a Ser or Ala residue at position 158, a glutamine at position 162, or a combination thereof, with the position numbering corresponding to positions of the wild-type tyrosyl tRNA synthetase. The cell can be lysed to produce a lysate that s purified by exposing the lysate to a purification matrix that selectively binds to the boronic amino acid residue.

Relatedly, the invention provides compositions that include a solid phase matrix covalently bound to a polypeptide through a boronic amino acid residue. The solid phase matrix can optionally comprise a saccharide resin. Optionally, the solid phase matrix can comprise a polyhydroxylated moiety, a saccharide, a polysaccharide, an n-methylglucamine moiety, or the like, bound to the boronic amino acid residue.

The invention also provides compositions that include a purified population of polypeptide molecules that each comprise a boronic amino acid, e.g., an aliphatic, aryl or heterocycle substituted boronic acid, a p-boronophenylalanine, an o-boronophenylalanine or an m-boronophenylalanine at a selected site. Such polypeptides can optionally be, e.g., at least 20 amino acid residues in length, at least 50 amino acid residues in length, at least 100 amino acid residues in length, or more than 100 amino acid residues in length. The polypeptide molecules in the population can optionally comprise an additional moiety bound to the boronic amino acid. The polypeptide molecules in the population can optionally comprise a therapeutic protein, an immunogen, an enzyme, a cell receptor ligand, a modulator of a serine protease, an inhibitor of a serine protease, a modulator of a glycosylated macromolecule, an inhibitor of a glycosylated macromolecule, a saccharide binding protein, an oligosaccharide binding protein, an antibody, an antibody fragment, a therapeutic antibody, an antibody or antibody fragment that specifically binds to a glycoprotein, an antibody that specifically binds to a serine protease, an antibody that specifically binds to a serum protease, a phage display protein, or a cancer cell ligand.

For example, the polypeptide molecules can optionally comprise or can optionally be homologous to an Aldosterone Receptor, an antibody or antibody fragment, Alpha-1 antitrypsin, Angiostatin, Antihemolytic factor, Apolipoprotein, Apoprotein, Atrial natriuretic factor, Atrial natriuretic polypeptide, Atrial peptide, a C—X—C chemokine, T39765, NAP-2, ENA-78, Gro-a, Gro-b, Gro-c, IP-10, GCP-2, NAP-4, SDF-1, PF4, MIG, Calcitonin, c-kit ligand, a cytokine, a CC chemokine, a corticosterone, estrogen receptor, Met, Monocyte chemoattractant protein-1, Monocyte chemoattractant protein-2, Monocyte chemoattractant protein-3, Monocyte inflammatory protein-1 alpha, Monocyte inflammatory protein-1beta, Mos, Myc, RANTES, 1309, R83915, R91733, HCC1, T58847, D31065, T64262, CD40, CD40 ligand, CD44, C-kit Ligand, Collagen, Colony stimulating factor (CSF), Complement factor 5a, Complement inhibitor, Complement receptor 1, epithelial Neutrophil Activating Peptide-78, GRO′γ, MGSA, GROβ, GROγ, MIP1-α, MIP1-β, MIP1-Δ, MCP-1, Epidermal Growth Factor (EGF), epithelial Neutrophil Activating Peptide, Erythropoietin (EPO), Exfoliating toxin, Factor IX, Factor VII, Factor VIII, Factor X, Fibroblast Growth Factor (FGF), Fibrinogen, Fibronectin, Fos, G-CSF, GM-CSF, Glucocerebrosidase, Gonadotropin, growth factor, growth factor receptor, Hyalurin, Hedgehog protein, Hemoglobin, Hepatocyte Growth Factor (HGF), Hirudin, Human serum albumin, ICAM-1, an ICAM-1 receptor, an LFA-1, LFA-1 receptor, an inflammatory protein, Insulin, Insulin-like Growth Factor (IGF), IGF-I, IGF-II, interferon, IFN-α, IFN-β, IFN-γ, interleukin, IL-1, IL-2, IL-3, IL-4, IL-5, IL-6, IL-7, IL-8, IL-9, IL-10, IL-11, IL-12, Jun, Keratinocyte Growth Factor (KGF), Lactoferrin, leukemia inhibitory factor, LDL receptor, Luciferase, Myb, Neurturin, Neutrophil inhibitory factor (NIF), oncostatin M, Osteogenic protein, oncogene product, Parathyroid hormone, PD-ECSF, PDGF, peptide hormone, progesterone receptor, Human Growth Hormone, p53, Pleiotropin, Protein A, Protein G, Pyrogenic exotoxin A, B, or C, Ras, Raf, Rel, Relaxin, Renin, a signal transduction protein, SCF/c-kit, Soluble complement receptor I, Soluble I-CAM 1, Soluble interleukin receptor, Soluble TNF receptor, Somatomedin, Somatostatin, Somatotropin, Streptokinase, Superantigen, Staphylococcal enterotoxin, SEA, SEB, SEC 1, SEC2, SEC3, SED, SEE, steroid hormone receptor, Superoxide dismutase, Tat, Testosterone Receptor, Toxic shock syndrome toxin, Thymosin alpha 1, Tissue plasminogen activator, tumor growth factor (TGF), TGF-α variants, TGF-β, a transcriptional activator protein, a transcriptional suppressor protein, Tumor Necrosis Factor, Tumor Necrosis Factor alpha, Tumor necrosis factor beta, Tumor necrosis factor receptor (TNFR), Urokinase, VLA-4 protein, VCAM-1 protein, and Vascular Endothelial Growth Factor (VEGEF).

Kits are also a feature of the invention. Kits can include any of the compositions herein, e.g., packaged in an appropriate container, along with instructional materials, e.g., to practice the methods of the invention.

Those of skill in the art will appreciate that the methods, kits and compositions provided by the invention can be used alone or in combination. For example, compositions comprising an aminoacyl tRNA synthetase that selectively recognizes a boronic amino acid can be used in the methods for incorporating a boronic amino acid into a target polypeptide. Alternately or additionally, these methods can be used to produce, e.g., a population of purified polypeptide molecules that each comprise a boronic amino acid at a selected site. One of skill will appreciate further combinations of the features of the invention noted herein.

### BRIEF DESCRIPTION OF THE DRAWINGS

FIG. 1A provides the structure of p-boronophenylalanine, a schematic depiction of its reduction to phenylalanine, and a schematic depiction of its oxidation to tyrosine. FIG. 1B illustrates the results of an experiment performed to determine the specificity and efficiency of p-boronophenylalanine incorporation into Z domain by MjtRNA^{Tyr}_{CUA}/B(OH)₂PheRS.

FIG. 2 provides proteins sequences for Z-domain and T4 lysozyme mutants into which p-boronophenylalanine was incorporated.

FIG. 3A depicts the results of ESI-TOF experiments performed to confirm the expected mass of boronate containing Z-domain (Z-domain-K7(p-boronophenylalanine)). FIG. 3B depicts the results of ESI-TOF experiments performed to confirm the expected mass of the oxidized tyrosine product. FIG. 3C depicts results of ESI-TOF experiments performed to confirm the expected mass of the reduced phenylalanine product.

FIG. 4A depicts the results of electrospray ionization time-of-flight experiments performed to confirm the expected mass of boronate containing T4 lysozyme (T4L-A82(p-boronophenylalanine)). FIG. 4B depicts the results of ESI-TOF experiments performed to confirm the expected mass of the H₂0₂-oxidized tyrosine product. FIG. 4C depicts results of ESI-TOF experiments performed to confirm the expected mass of the potassium peroxymonosulfate-oxidized tyrosine product.

FIG. 5A provides structures of glucamine (2), fluorescein-glucamine probe (3), and immobilized glucamine affinity purification resin (4; XUS43594.00 Dow Chemicals). FIG. 5B depicts the selective fluorescent labeling of Z-domain-K7(p-boronophenylalanine) over Z-domain-K7Y with fluorescein-NHS-glucamine. FIGS. 5C and 5D show the results of affinity purification of Z-domain-K7(p-boronophenylalanine) and Z-domain-K7Y, respectively, using nickel-NTA resin or boronate affinity resin

FIG. 6A depicts the reconstructed ESI-TOF of a Z-domain glucamine resin sorbitol elution fraction. FIG. 6B depicts the reconstructed ESI-TOF of a Z-domain glucamine resin hydrogen peroxide elution fraction.

FIG. 7A provides the structure of Compound 5. FIG. 7B shows the results of Suzuki coupling of 50 μM T4L-A82(p-boronophenylalanine) to 1 mM reporter molecule (Compound 5).

### DETAILED DESCRIPTION

The invention provides orthogonal systems for genetically encoding boronic amino acids into proteins of interest. Boronic amino acids that can be genetically encoded include, e.g., aliphatic, aryl or heterocycle substituted boronic acids, e.g., p-boronophenylalanine (bPh), o-boronophenylalanine, m-boronophenylalanine, or the like. A variety of orthogonal components that operate together in cells to encode boronic amino acids into proteins of interest are provided. For example, several new synthetases that are specific for p-boronophenylalanine are provided in the sequence listing herein. These and related synthetases, in conjunction with their cognate tRNAs, provide for site-specific boronic amino acid incorporation in a cell in response to a selector codon. The synthetases and tRNAs, which are orthogonal to the cell, incorporate the boronic amino acid in response to the selector codon, which, in turn, is engineered into a nucleic acid that encodes a protein of interest. For an overview of orthogonal translation systems generally, see, Wang, et al., (2006) “Expanding the Genetic Code.” Annu Rev Biophys Biomol Struct 35: 225-249; Wang and Schultz, (2005) “Expanding the Genetic Code.” Angewandte Chemie Int Ed 44: 34-66; Xie and Schultz, (2005) “An Expanding Genetic Code.” Methods 36: 227-238; Xie and Schultz, (2005) “Adding Amino Acids to the Genetic Repertoire.” Curr Opinion in Chemical Biology 9: 548-554. See also, the section below entitled “ORTHOGONAL TRNA/AMINOACYL-TRNA SYNTHETASES OF THE INVENTION.”

The incorporation of boronic amino acids into proteins is highly useful, both with respect to the many diverse applications for the boronic amino acid moiety in proteins, and also with respect to the wide variety of different proteins that the boronic amino acid can be incorporated into. Proteins that comprise boronic acids, such as bPh, have several unique chemical properties, including the ability to participate in transition metal catalyzed reactions, oxidation/reduction reactions, and boronic ester equilibrium.

Isotopes of boron, when incorporated into therapeutic proteins can also provide selective cancer treatments, e.g., through boron neutron capture therapies. Boronate capture and release from solid phase sugar resins also allows for one step purification of boronate containing proteins, without the need for traditional purification tags such as 6×His tags, streptavidin tags, or fusion proteins. This is a significant advantage over tag-based purification methods, as any influence of the tag on the ultimate activity of the purified protein is eliminated. The ability of bPh or other boronic acids to be oxidized or reduced to phenylalanine or tyrosine provides “scarless” purification of native protein sequences, free of unwanted modifications.

The unusual ability of boronate to bind sugars with high affinity also provides for the creation of proteins (including, e.g., antibodies) that covalently bind oligosaccharides, a characteristic not found in the 20 standard amino acids. Boronate containing proteins also provide a new class of protein-based, selective serum and/or serine protease inhibitors.

### Applications of the Borono Group in Proteins

The invention provides new orthogonal tRNA/aminoacyl tRNA synthetase pairs that provide for the selective incorporation of boronic acids, e.g., para-boronophenylalanine (bPh), into proteins in cells, in response to a selector codon such as an amber (TAG) stop codon. In contrast to chemosynthetic approaches for making peptides with boronate moieties (e.g., dipeptides), proteins of the invention can be full-length proteins, e.g., 10, 20, 50, 100, or more amino acid residues in length.

Applications for boronic amino acids that are incorporated into proteins include participation in any of a variety of highly useful chemical reactions that can be used in inter or intra molecular coupling reactions, e.g., to site-specifically attach any of a wide variety of constituents of interest to a protein, to stabilize the protein in a selected conformation, to add oligosaccharide specific binding activity to the protein (e.g., to provide for one-step purification on a saccharide matrix), or the like. The boronic amino acid can be used, e.g., for “scarless” protein purification of essentially any recombinant protein of interest, e.g., as an improvement over ubiquitous protein tag-based purification methods. In addition to such fundamental uses in protein engineering and purification, the boronic acid moiety can, itself, be used as a therapeutic agent.

Boronate-Mediated Chemical Reactions

The addition of the boronate moiety onto a protein surface imparts new bio-orthogonal chemistry to proteins, which is used in the selective modification and purification of proteins, for biomolecular probe addition, and in the design of proteins, including for use in therapeutic proteins, immunogens, enzymes, cell receptor ligands, modulators of a serine proteases, inhibitors of a serine proteases, modulators of a glycosylated macromolecules, inhibitors of a glycosylated macromolecules, saccharide binding proteins, oligosaccharide binding proteins, antibodies, antibody fragments, therapeutic antibodies, antibodies or antibody fragments that specifically bind to an oligosaccharide or glycoprotein, antibodies that specifically binds to a serine proteases, antibodies that specifically binds to a serum protease, a phage display protein, or a cancer cell ligand.

One practical application of proteins comprising a boronic amino acid residue includes the covalent attachment of aryl iodides to bPh through palladium catalyzed Suzuki couplings. For a description of this chemistry, see, e.g., Miyaura and Suzuki (1995) “Palladium-Catalyzed Cross-Coupling Reactions of Organoboron Compounds,” Chemical Reviews 95: 2457 and Suzuki (1999) “Recent advances in the cross-coupling reactions of organoboron derivatives with organic electrophiles, 1995-1998,” Journal of Organometallic Chemistry 576:147. Aryl iodides are reactive groups with a variety of uses in organometallic chemistry, including silylation, aminocarbonylation, Heck Arylation, vinylation, cross-coupling with aryl acetylenes, and many others.

The boronate group is similarly useful in copper catalyzed heteroatom alkylation reactions (Chan, et al. (2003) “Copper promoted C—-N and C—-O bond cross-coupling with phenyl and pyridylboronates,” Tetrahedron Letters 44:3863), asymmetric reductions (Huang, et al (2000) “Asymmetric reduction of acetophenone with borane catalyzed by chiral oxazaborolidinone derived from L-a-amino acids,” Synthetic Communications 30:2423), Diels-Alder reactions (Ishihara and Yamamoto (1999) “Arylboron Compounds as Acid Catalysts in Organic Synthetic Transformations,” European Journal of Organic Chemistry 527), as well as a variety of other transformations.

Boronic acid residues can also be used to form reversible boronic esters with alcohols, diols (including sugars), amino-alcohols, and diamine containing compounds. For example, boronic acids form reversible covalent complexes with diols. For an early description of this chemistry, see Lorand and Edwards, (1959) “Polyol Complexes and Structure of the Benzeneboronate Ion,” Journal of Organic Chemistry 24:769. Reversible complexes can also be formed with aminoalcohols (Springsteen, et al. (2001) “The Development of Photometric Sensors for Boronic Acids,” Bioorganic Chemistry 29:259), amino acids (Mohler and Czarnik, “Amino acid Chelative complexation by an Arylboronic Acid,” Journal of the American Chemical Society 116:2233; Mohlerand Czarnik (1993) “Alpha-Amino-Acid Chelative Complexation by an Arylboronic Acid,” Journal of the American Chemical Society 115: 7037) alkoxides (Cammidge and Crépy (2004) “Synthesis of chiral binaphthalenes using the asymmetric Suzuki reaction,” Tetrahedron 60:4377.), and hydroxamic acids (Lamandé, et al. (1980) “Structure et acidite de composes a atome de bore et de phosphore hypercoordonnes,” Journal of Organometallic Chemistry 329). Similarly, the boronate containing protein can be modified with biophysical reporters, PEGs and other synthetic groups.

Using these chemistries, any of a wide variety of biomolecular probes can be bound at boronic amino acid sites, including saccharides, oligosaccharides, dyes, labels, functional groups (e.g., for surface immobilization, including, e.g., silane mediated surface attachment), organic moieties, proteins, peptides, nucleic acids, lipids, nanomaterials, particles, magnetic particles, and many others.

Selective Saccharide Recognition

Boronic acids have been used in the synthesis of ligands for the selective recognition of sugars (James, et al. (1996) “Saccharide Sensing with Molecular Receptors Based on Boronic Acid,” Angewandte Chemie-International Edition in English 35:1910; James, et al. (1995) “Chiral discrimination of monosaccharides using a fluorescent molecular sensor,” Nature 374:345; Wang, et al. (2002) “Boronic Acid-Based Sensors,” Current Organic Chemistry 6:1285). In the present invention, proteins comprising boronic acids can be used for the selective recognition of saccharide (e.g., oligosaccharide) containing moieties. The ability to selectively bind saccharides is not found in the 20 natural amino acids; thus, the invention provides a convenient new system for producing proteins that have oligosaccharide binding activity. The boronic amino acids are used to target proteins comprising them to a wide variety of biomolecules and biostructures, e.g., for labeling, to serve as a targeting agent during therapy, or the like. Proteins that comprise the boronic amino acids can include, e.g., recombinant antibodies or antibody ligands, cell surface proteins such as receptors or cell surface/receptor ligands, etc. These boronic acid-containing proteins can bind to cognate antibodies, surface proteins, receptors, or ligands, etc., e.g., where these cognate components comprise a saccharide or oligosaccharide moiety.

Scarless Purification Using Saccharide Binding

In one useful aspect, proteins that contain bPH or other boronic amino acids can be covalently attached to solid phase sugar resins, providing for one step protein purification. To release the protein from the resin, the boronic acid moiety can be chemically oxidized to tyrosine, or, alternately, reduced to phenylalanine, providing a procedure termed “scarless” protein purification. The protein can also be eluted, e.g., with an appropriate saccharide, if the borono group is to be retained in the purified protein.

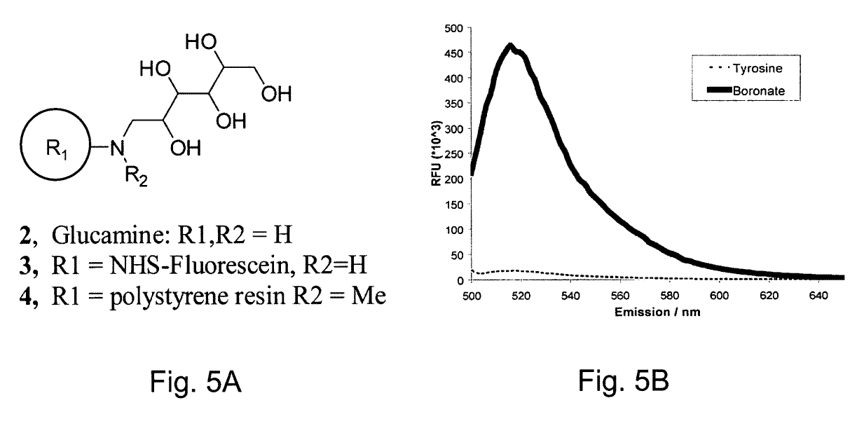

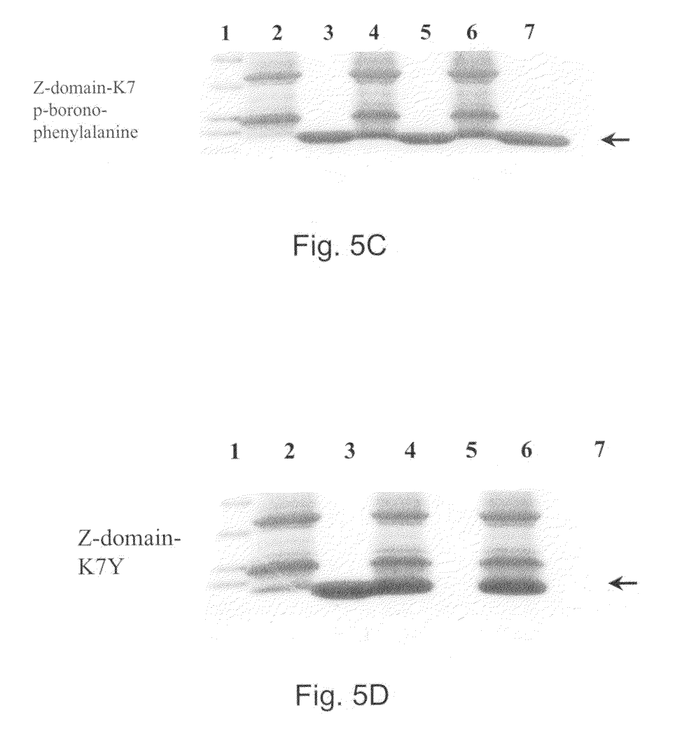

For example, boronic acids form strong reversible covalent interactions with polyhydroxylated compounds in aqueous solutions at physiological pH (Lorand and Edwards, (1959) “Polyol Complexes and Structure of the Benzeneboronate Ion,” Journal of Organic Chemistry 24: 769; Springsteen and Wang (2002) “A detailed examination of boronic acid-diol complexation,” Tetrahedron 58:5291). The interaction of boronates with sorbitol and glucamine (FIG. 5A, Compound 2; R1, R2=H), for example, are of high affinity and provide a useful method to selectively label proteins under conditions that are relatively benign to biological systems. As described in detail in Example 1 below, the ability of polyhydroxylated molecules to bind boronate containing proteins was tested and confirmed.

As also described in detail in Example 1, this chemistry was used to provide a new general strategy for the affinity purification of boronic acid-containing proteins from cell lysates. N-methylglucamine conjugated polystyrene resin was used for the affinity purification of a p-boronophenylalanine residue (other polyhydroxylated conjugates could have similarly been used). The resin was split and protein was eluted by the addition of excess sorbitol (1 M) or by oxidation of the boronate to tyrosine, using excess hydrogen peroxide. The protein was isolated in high purity using either elution method. In fact, purification yields were comparable to those achieved using Ni-NTA/His Tag purification. Mass spectral analysis of purified protein showed that hydrogen peroxide elution fully converted the p-boronophenylalanine to tyrosine. The sorbitol elution isolated protein containing the boronic acid residue. Due to the ability of p-boronophenylalanine to be oxidized or reduced to tyrosine or phenylalanine, respectively, this methodology allows the purification of native protein sequences.

In this “scarless” protein purification procedure, a selector codon is encoded into a protein in place of a codon for tyrosine or phenylalanine, resulting in a boronic amino acid being incorporated into an encoded protein during translation, using orthogonal components in a cell or other translation system. The boronic amino acid is converted back to tyrosine or phenylalanine during purification by oxidation, or reduction, respectively.

In a single simple purification step, a protein can be isolated, based on the unique chemistry of the unnatural amino acid alone, without the need for commonly used protein affinity tags such as 6×His or fusion proteins. Selective oxidation or reduction yields native placement of a tyrosine or phenylalanine residue at the boronic acid site, ultimately yielding a native protein sequence, with no modification remaining from the purification process.

The sorbitol elution also demonstrates that, where a purified protein comprising a borono residue is desired, this can also be produced by a one-step affinity purification.

Boronic Serine Protease Inhibitors

Serine proteases or “serine endopeptidases” are a well-characterized large class of protease enzymes that comprise serine at the active site of the protein. Serine proteases are physiologically regulated by cognate serine protease inhibitors (e.g., serpins), which typically inhibit the enzymes selectively, e.g., when they are no longer needed for protease function by the cell or organism. Serine protease inhibitors control processes such as coagulation and inflammation and are in use as therapeutic agents in a variety of contexts. Serine protease misregulation can lead to a variety of clinical disorders, e.g., blood clotting disorders (e.g., Antiplasmin deficiency or Antithrombin deficiency), high blood pressure (e.g., angiotensinogen misregulation), emphysema (e.g., from Alpha-1-antitrypsin deficiency), edema (caused, e.g., by C1INH deficiency), thrombosis (e.g., from antithrombin deficiency), several cancers, liver cirrhosis (caused, e.g., by antitrypsin polymerization) and many other diseases and conditions. Serine protease inhibitors are also used in a variety of other diverse contexts, e.g., for use in structural biology (serpins undergo a unique structural shift as they bind to a serine protease, which is relevant to Alzheimer's disease and prion mediated diseases), and even as insecticides (parathion is an acetylcholinesterase inhibitor), as well as many others.

Boronic acid containing proteins can be used to form covalent inhibitors of serine proteases. For example, the invention provides a site specific mechanism for generating boronic acid containing proteins. Known serine protease inhibitors (e.g., various serpins) can be modified to include boronic amino acids, which can be used to covalently bind to the serine residue at the active site, disabling the protease. More generally, a ligand that mimics the structure of a target of a given protease, or, e.g., an antibody that binds the protease, can be modified or designed to comprise a boronic amino acid, thereby providing an inhibitor that covalently binds to the serine protease. If the boronic acid is proximal to the active site of the protease when bound, the boronic acid can form a covalent linkage with the serine at the active site, disabling the enzyme.

Over 1000 serine protease inhibitors have been identified and can be adapted to the invention by adding a boronic amino acid residue. For a description of the serpins, see, e.g., Irving, et al. (2000) “Phylogeny of the Serpin Superfamily: Implications of Patterns of Amino Acid Conservation for Structure and Function,” Genome Res. 10:1845-64; Irving, et al. (2002), “Serpins in prokaryotes,” Mol Biol Evol 19 (11): 1881-90. Non-limiting examples of serine proteases and serine protease inhibitors that can be modified to incorporate a boronic amino acid include: Chymotrypsin/alpha-1-antichymotrypsin; Complement factor C1s/C1 Inhibitor (C1INH); Elastase/alpha-1-antitrypsin; Clotting factor 10 (X)/antithrombin III; Thrombin/antithrombin III; Plasmin/alpha-2-antiplasmin; and Trypsin/pancreatic trypsin inhibitor.

For additional details regarding the use of boronic acids as serine protease inhibitors, see also, Adams, et al. (1998) “Potent and selective inhibitors of the proteasome: Dipeptyidyl boronic acids,” Bioorganic & Medicinal Chemistry Letters 8:333; Weston, et al. (1998) “Structure-Based Enhancement of Boronic Acid Inhibitors of AmpC b-Lactamase,” Journal of Medicinal Chemistry 41:4577; Yang, et al. (2003) “Boronic acid compounds as potential pharmaceutical agents,” Medicinal Research Reviews 23:346; and Matthews, et al. (1975) “X-ray crystallographic study of boronic acid adducts with subtilisin BPN′ (Novo). A model for the catalytic transition state,” Journal of Biological Chemistry 250:7120).

Most serum proteases are also serine proteases; in any case, similar considerations apply to the general class of serum proteases and their inhibitors as for those discussed above for serine proteases/inhibitors.

Target Cell Labeling and Therapy

Cells can be targeted by the boronic acid containing proteins of the invention (or proteins derived from boronic acid containing proteins by one or more chemical reaction performed on the boronate moiety). This targeting can take any of the usual forms of cell targeting by proteins, e.g., mediated through specific binding to cell receptors or other cell associated molecules by the proteins of the invention. Boronic amino acids can also themselves be used to target, e.g., saccharide moieties on cells, essentially as discussed above. The boronic acid modified proteins (or derivatives thereof) can be used to label one or more component of the cell, or to have a therapeutic effect on the cell or for an organism (e.g., patient) that comprises the cell.

In one useful embodiment, boronates can be used as boron neutron capture agents to kill target cells such as tumor cells (Kinashi, et al. (2002) “Mutagenic effect of borocaptate sodium and boronophenylalanine in neutron capture therapy,” International Journal of Radiation Oncology Biology Physics 54:562). In the context of the invention, this is particularly useful, because a variety of proteins are known to be specifically bound or internalized by target cells. These proteins can be engineered to include one or more boronic acid. Once they are localized to the target cell population, neutron capture be used to kill the target cell. For example, a localized field of lethal a particles can be produced upon neutron irradiation of the boronoate moiety, thereby killing the tumor or other target cell.

Similarly, proteins that are modified through a boronate moiety to include any of the useful features noted herein, e.g., biomolecular probes, saccharides, diols, reactive groups, PEG, fusion protein moieties, or the like can be targeted to the cell, relying either on the usual interactions between a protein and its target, or through a unique activity (e.g., oligosaccharide binding) of the borono group (or of a group added to the protein through the boronic acid mediated chemistries noted herein). Features of the boronate moiety can also facilitate delivery of the protein, e.g., PEG binding can improve serum half-life of the protein, enabling it to reach its target, or simply to have a longer activity half-life in a patient.

Essentially any protein can be engineered according to the invention to include a boronic amino acid, for therapeutic use in relation to a target cell of interest. Examples of appropriate proteins to be engineered with one or more boronic amino acid for this aspect of the invention includes target cell associated or specific ligands, receptor ligands, antibodies that bind to the target cells (e.g., antibodies that bind tumor markers), and the like. For example, tumor-specific antigens (TSA) are specific to tumors and not found in normal tissue, while tumor-associated antigens (TAA) are found in both tumor cells and normal tissues. Tumor-associated carbohydrate antigens are another well described type of tumor antigen (and, as noted herein, boronic amino acids can be used to target carbohydrates). Any or all of these biologically relevant targets can be bound by a boronic acid containing protein of the invention, including for labeling or for targeted cell death, e.g., by neutron capture, or mediated by an activity of a moiety attached to the protein using the borono chemistries noted herein.

Diseases that can be treated by targeting relevant cells include cancer, autoimmune diseases, lupus, infectious diseases, bacterially mediated diseases, tuberculosis, leprosy, sexually transmitted diseases, virally mediated diseases, HIV infection, AIDS, herpes virus mediated diseases, poliovirus mediated diseases, parasite infections, Plasmodium infections, malaria, prion-mediated diseases, and many others.

For example, literally thousands of tumor associated and specific antigens are known and well described in the literature. These antigens can be targeted by antibodies or other ligands that bind to the antigens, engineered according to the invention (e.g., to include a boronic amino acid, or a product of a reaction with the boronic amino acid). Cancers that express TSA or TAA include, but are not limited to, biliary tract cancer; bone cancer, brain cancer (e.g., gliomas); breast cancer; cervical or other reproductive system cancers (uterine, ovarian, testicular, etc.); choriocarcinoma; colon cancer; endometrial cancer; esophageal cancer; gastric cancer (e.g., stomach cancer); intraepithelial neoplasms; lymphomas; liver cancer; lung cancer (e.g. small cell and non-small cell); melanomas; neuroblastomas; oral cancer; ovarian cancer; pancreas cancer; prostate cancer; rectal cancer; sarcomas; skin cancer; thyroid cancer; and renal cancer, as well as many other well described and known carcinomas and sarcomas.

Example polypeptide molecules that can be modified to include a boronic amino acid include or are homologous to known polypeptides such as Aldosterone Receptor, an antibody or antibody fragment, Alpha-1 antitrypsin, Angiostatin, Antihemolytic factor, Apolipoprotein, Apoprotein, Atrial natriuretic factor, Atrial natriuretic polypeptide, Atrial peptide, a C-X-C chemokine, T39765, NAP-2, ENA-78, Gro-a, Gro-b, Gro-c, IP-10, GCP-2, NAP-4, SDF-1, PF4, MIG, Calcitonin, c-kit ligand, a cytokine, a CC chemokine, a corticosterone, estrogen receptor, Met, Monocyte chemoattractant protein-1, Monocyte chemoattractant protein-2, Monocyte chemoattractant protein-3, Monocyte inflammatory protein-1 alpha, Monocyte inflammatory protein-1 beta, Mos, Myc, RANTES, 1309, R83915, R91733, HCC1, T58847, D31065, T64262, CD40, CD40 ligand, CD44, C-kit Ligand, Collagen, Colony stimulating factor (CSF), Complement factor 5a, Complement inhibitor, Complement receptor 1, epithelial Neutrophil Activating Peptide-78, GRO′γ, MGSA, GROβ, GROγ, MIP1-α, MIP1-β, MIP1-Δ, MCP-1, Epidermal Growth Factor (EGF), epithelial Neutrophil Activating Peptide, Erythropoietin (EPO), Exfoliating toxin, Factor IX, Factor VII, Factor VIII, Factor X, Fibroblast Growth Factor (FGF), Fibrinogen, Fibronectin, Fos, G-CSF, GM-CSF, Glucocerebrosidase, Gonadotropin, growth factor, growth factor receptor, Hyalurin, Hedgehog protein, Hemoglobin, Hepatocyte Growth Factor (HGF), Hirudin, Human serum albumin, ICAM-1, an ICAM-1 receptor, an LFA-1, LFA-1 receptor, an inflammatory protein, Insulin, Insulin-like Growth Factor (IGF), IGF-I, IGF-II, interferon, IFN-α, IFN-β, IFN-γ, interleukin, IL-1, IL-2, IL-3, IL-4, IL-5, IL-6, IL-7, IL-8, IL-9, IL-10, IL-11, IL-12, Jun, Keratinocyte Growth Factor (KGF), Lactoferrin, leukemia inhibitory factor, LDL receptor, Luciferase, Myb, Neurturin, Neutrophil inhibitory factor (NIF), oncostatin M, Osteogenic protein, oncogene product, Parathyroid hormone, PD-ECSF, PDGF, peptide hormone, progesterone receptor, Human Growth Hormone, p53, Pleiotropin, Protein A, Protein G, Pyrogenic exotoxin A, B, or C, Ras, Raf, Rel, Relaxin, Renin, a signal transduction protein, SCF/c-kit, Soluble complement receptor I, Soluble I-CAM 1, Soluble interleukin receptor, Soluble TNF receptor, Somatomedin, Somatostatin, Somatotropin, Streptokinase, Superantigen, Staphylococcal enterotoxin, SEA, SEB, SEC 1, SEC2, SEC3, SED, SEE, steroid hormone receptor, Superoxide dismutase, Tat, Testosterone Receptor, Toxic shock syndrome toxin, Thymosin alpha 1, Tissue plasminogen activator, tumor growth factor (TGF), TGF-α variants, TGF-β, a transcriptional activator protein, a transcriptional suppressor protein, Tumor Necrosis Factor, Tumor Necrosis Factor alpha, Tumor necrosis factor beta, Tumor necrosis factor receptor (TNFR), Urokinase, VLA-4 protein, VCAM-1 protein, and Vascular Endothelial Growth Factor (VEGEF).

For therapeutic or other in vivo applications (e.g., using the proteins of the invention as labels), routes of administration of the proteins of the invention depend on the application. Local injection into or near the target cell can be used, as can systemic delivery, e.g., via intravenous injection. In general, administration is by any of the routes normally used for introducing a composition into ultimate contact with cells or tissues of interest. Practitioners can select an administration route of interest based on the target for delivery. Circulating target cells such as T-cells or other blood cells, can also be exposed to the proteins of the invention ex vivo, and later returned to the patient intravenously.

The dose of therapeutic protein of the invention (antibody, etc.) administered to a patient, in the context of the present invention, is sufficient to effect a beneficial therapeutic response in the patient over time. The dose is determined by the efficacy of the particular composition and the activity, stability or serum half-life of the composition, and the condition of the patient, as well as the body weight or surface area of the patient to be treated. Here again, reference to available therapies that include delivery of similar proteins (except lacking the boronic amino acid, or derivative thereof) in vivo, or ex vivo can be used to estimate dosages.

Intra-Molecular Protein Stabilization

The methods can include forming a covalent bond between the boronic acid and an additional residue (e.g., serine or threonine) of the target polypeptide, thereby stabilizing the conformation of the polypeptide. That is, the boronic amino acid can complex with one or more reactive serine, threonine or tyrosine residue(s) that complex with the boronic acid group, locking the protein of interest into an active (or an inactive) conformer. This can be useful for structural studies (e.g., crystallizations, etc.), for regulating protein activity, and, e.g., for regulating the immunogenicity of the protein.

For example, immunogens that comprise particular protein conformers can display enhanced immunogenicity, e.g., where a subset of all possible immunogen conformers are displayed to the immune system (e.g., for antibody production). Similarly, for many secretory and integral membrane proteins, biosynthesis and folding is a highly heterogeneous processes; the ability to lock proteins into one conformation can influence both protein function and degradation pathways for the protein (degradation pathways are sometimes conformer specific). In fact, the differential display of different polypeptide conformers underlies a variety of disease states, which can be assessed or treated using antibodies that are specific for a given conformer. See, e.g., 20070015211 “Conformer-specific antibodies and method of use, thereof” by Lingappa.

### Orthogonal TRNA/Aminoacyl-TRNA Synthetases of the Invention

The invention includes orthogonal components that are capable of selectively incorporating a boronic amino acid in response to a selector codon. For example 1BF6, 1BF9, 1BE3, 1BF10, 1BF12, 1BG10, or 1BG11 all incorporate p-boronophenylalanine. Given Applicants discovery that RS specific for the borono group can be produced, it is expected that aliphatic, aryl or heterocycle substituted boronic acids, e.g., p-boronophenylalanine, o-boronophenylalanine, and m-boronophenylalanine can be produced using essentially similar techniques. Specific details regarding production of 1BF6, 1BF9, 1BE3, 1BF10, 1BF12, 1BG10, or 1BG11 can be found in Example 1.

In general, in order to add additional unnatural amino acids to the genetic code, new orthogonal pairs comprising an aminoacyl-tRNA synthetase and a suitable tRNA are needed that can function efficiently in the host translational machinery, but that are “orthogonal” to the translation system at issue, meaning that it functions independently of the synthetases and tRNAs endogenous to the translation system. Desired characteristics of the orthogonal pair include tRNA that decode or recognize only a specific codon, e.g., a selector codon, e.g., and amber stop codon, that is not decoded by any endogenous tRNA, and aminoacyl-tRNA synthetases that preferentially aminoacylate, or “charge”, its cognate tRNA with a specific unnatural amino acid (e.g., an aliphatic, aryl or heterocycle substituted boronic acid, e.g., p-boronophenylalanine, o-boronophenylalanine, or m-boronophenylalanine). The O-tRNA is also not typically aminoacylated, or is very poorly aminoacylated, i.e., “charged,” by endogenous synthetases. For example, in an E. coli host system, an orthogonal pair will include an aminoacyl-tRNA synthetase that does not cross-react with any of the endogenous tRNAs, e.g., of which there are 40 endogenous in E. coli, and an orthogonal tRNA that is not aminoacylated by any of the endogenous synthetases, e.g., of which there are 21 in E. coli. The term “cognate” refers to components that function together, or have some aspect of specificity for each other, e.g., an orthogonal tRNA and an orthogonal aminoacyl-tRNA synthetase.

The general principles for the production of orthogonal translation systems that are suitable for making proteins that comprise one or more desired unnatural amino acid are known in the art, as are the general methods for producing orthogonal translation systems. For example, see International Publication Numbers WO 2002/086075, entitled “METHODS AND COMPOSITION FOR THE PRODUCTION OF ORTHOGONAL tRNA-AMINOACYL-tRNA SYNTHETASE PAIRS;” WO 2002/085923, entitled “IN VIVO INCORPORATION OF UNNATURAL AMINO ACIDS;” WO 2004/094593, entitled “EXPANDING THE EUKARYOTIC GENETIC CODE;” WO 2005/019415, filed Jul. 7, 2004; WO 2005/007870, filed Jul. 7, 2004; WO 2005/007624, filed Jul. 7, 2004; WO 2006/110182, filed Oct. 27, 2005, entitled “ORTHOGONAL TRANSLATION COMPONENTS FOR THE VIVO INCORPORATION OF UNNATURAL AMINO ACIDS” and WO 2007/103490, filed Mar. 7, 2007, entitled “SYSTEMS FOR THE EXPRESSION OF ORTHOGONAL TRANSLATION COMPONENTS IN EUBACTERIAL HOST CELLS.” Each of these applications is incorporated herein by reference in its entirety. For discussion of orthogonal translation systems that incorporate unnatural amino acids, and methods for their production and use, see also, Wang and Schultz, (2005) “Expanding the Genetic Code.” Angewandte Chemie Int Ed 44: 34-66; Xie and Schultz, (2005) “An Expanding Genetic Code.” Methods 36: 227-238; Xie and Schultz, (2005) “Adding Amino Acids to the Genetic Repertoire.” Curr Opinion in Chemical Biology 9: 548-554; and Wang, et al., (2006) “Expanding the Genetic Code.” Annu Rev Biophys Biomol Struct 35: 225-249; Deiters, et al, (2005) “In vivo incorporation of an alkyne into proteins in Escherichia coli.” Bioorganic & Medicinal Chemistry Letters 15:1521-1524; Chin, et al., (2002) “Addition of p-Azido-L-phenylalanine to the Genetic Code of Escherichia coli.” J Am Chem Soc 124: 9026-9027; and International Publication No. WO2006/034332, filed on Sep. 20, 2005, the contents of each of which are incorporated by reference in their entirety. Additional details are found in U.S. Pat. No. 7,045,337; No. 7,083,970; No. 7,238,510; No. 7,129,333; No. 7,262,040; No. 7,183,082; No. 7,199,222; and No. 7,217,809.

Orthogonal Translation Systems

Orthogonal translation systems generally comprise cells, e.g., prokaryotic cells such as E. coli, or eukaryotic cells such as yeast, plant, insect, or mammalian cells that include an orthogonal tRNA (O-tRNA), an orthogonal aminoacyl tRNA synthetase (O-RS), and an unnatural amino acid, e.g., a p-boronophenylalanine or other boronic amino acid, where the O-RS aminoacylates the O-tRNA with the unnatural amino acid, e.g., p-boronophenylalanine, etc. An orthogonal pair of the invention can include an O-tRNA, e.g., a suppressor tRNA, a frameshift tRNA, or the like, and a cognate O-RS. The orthogonal systems of the invention, which typically include O-tRNA/O-RS pairs, can comprise a cell or a cell-free environment. In addition to multi-component systems, the invention also provides novel individual components, for example, several novel orthogonal aminoacyl-tRNA synthetase polypeptides, e.g., those in the sequence listing, and the polynucleotides that encodes these polypeptides, e.g., as shown in the sequence listing.

In general, when an orthogonal pair recognizes a selector codon and loads an amino acid in response to the selector codon, the orthogonal pair is said to “suppress” the selector codon. That is, a selector codon that is not recognized by the translation system's, e.g., the E. coli, yeast, mammalian, etc. cell's, endogenous machinery is not ordinarily charged, which results in blocking production of a polypeptide that would otherwise be translated from the nucleic acid. In an orthogonal pair system, the O-RS aminoacylates the O-tRNA with a specific unnatural amino acid, e.g., p-boronophenylalanine. The charged O-tRNA recognizes the selector codon and suppresses the translational block caused by the selector codon.

In some aspects, an O-tRNA of the invention recognizes a selector codon and includes at least about, e.g., a 45%, a 50%, a 60%, a 75%, a 80%, or a 90% or more suppression efficiency in the presence of a cognate synthetase in response to a selector codon as compared to the suppression efficiency of an O-tRNA comprising or encoded by a polynucleotide sequence as set forth in the sequence listing herein.

In some embodiments, the suppression efficiency of the O-RS and the O-tRNA together is about, e.g., 5 fold, 10 fold, 15 fold, 20 fold, or 25 fold or more greater than the suppression efficiency of the O-tRNA lacking the O-RS. In some aspect, the suppression efficiency of the O-RS and the O-tRNA together is at least about, e.g., 35%, 40%, 45%, 50%, 60%, 75%, 80%, or 90% or more of the suppression efficiency of an orthogonal synthetase pair as set forth in the sequence listings herein.

The translation system, e.g., an E. coli, yeast, mammalian, etc. cell, uses the O-tRNA/O-RS pair to incorporate the unnatural amino acid, e.g., p-boronophenylalanine, etc., into a growing polypeptide chain, e.g., via a nucleic acid that comprises a polynucleotide that encodes a polypeptide of interest, where the polynucleotide comprises a selector codon that is recognized by the O-tRNA. In certain preferred aspects, the cell can include one or more additional O-tRNA/O-RS pairs, where the additional O-tRNA is loaded by the additional O-RS with a different unnatural amino acid. For example, one of the O-tRNAs can recognize a four base codon and the other O-tRNA can recognize a stop codon. Alternately, multiple different stop codons, multiple different four base codons, multiple different rare codons and/or multiple different non-coding codons can be used in the same coding nucleic acid. For further details regarding available O-RS/O-tRNA cognate pairs and their use, see, e.g., the references noted above.

As noted, in some embodiments, there exist multiple O-tRNA/O-RS pairs in translation system, which allow incorporation of more than one unnatural amino acid into a polypeptide. For example, the translation system can further include an additional different O-tRNA/O-RS pair and a second unnatural amino acid, where this additional O-tRNA recognizes a second selector codon and this additional O-RS preferentially aminoacylates the O-tRNA with the second unnatural amino acid. For example, a cell that includes an O-tRNA/O-RS pair, where the O-tRNA recognizes, e.g., an amber selector codon, can further comprise a second orthogonal pair, where the second O-tRNA recognizes a different selector codon, e.g., an opal codon, an ochre codon, a four-base codon, a rare codon, a non-coding codon, or the like. Desirably, the different orthogonal pairs are derived from different sources, which can facilitate recognition of different selector codons.

In certain embodiments, translation systems can comprise a cell, such as an E. coli or other bacterial cell, yeast, mammalian or other eukaryotic cell, that includes an orthogonal tRNA (O-tRNA), an orthogonal aminoacyl-tRNA synthetase (O-RS), an unnatural amino acid, e.g., an aliphatic, aryl or heterocycle substituted boronic acids, e.g., p-boronophenylalanine, o-boronophenylalanine, or m-boronophenylalanine, and a nucleic acid that comprises a polynucleotide that encodes a polypeptide of interest, where the polynucleotide comprises the selector codon that is recognized by the O-tRNA. Although orthogonal translation systems, e.g., translation systems comprising an O-RS, an O-tRNA and an unnatural amino acid, e.g., p-boronophenylalanine, etc., can utilize cultured cells to produce proteins having unnatural amino acids, it is not intended that an orthogonal translation system of the invention require an intact, viable cell. For example, a orthogonal translation system can utilize a cell-free system in the presence of a cell extract. Indeed, the use of cell free, in vitro transcription/translation systems for protein production is a well established technique. Adaptation of these in vitro systems to produce proteins having unnatural amino acids using orthogonal translation system components described herein is well within the scope of the invention.

The O-tRNA and/or the O-RS can be naturally occurring or can be, e.g., derived by mutation of a naturally occurring tRNA and/or RS, e.g., by generating libraries of tRNAs and/or libraries of RSs, from any of a variety of organisms and/or by using any of a variety of available mutation strategies. For example, one strategy for producing an orthogonal tRNA/aminoacyl-tRNA synthetase pair involves importing a tRNA/synthetase pair that is heterologous to the system in which the pair will function from a source, or multiple sources, other than the translation system in which the tRNA/synthetase pair will be used. The properties of the heterologous synthetase candidate include, e.g., that it does not charge any host cell tRNA, and the properties of the heterologous tRNA candidate include, e.g., that it is not aminoacylated by any host cell synthetase. In addition, the heterologous tRNA is orthogonal to all host cell synthetases. A second strategy for generating an orthogonal pair involves generating mutant libraries from which to screen and/or select an O-tRNA or O-RS. These strategies can also be combined.

Orthogonal tRNA (O-tRNA)

An orthogonal tRNA (O-tRNA) of the invention desirably mediates incorporation of an unnatural amino acid into a protein that is encoded by a polynucleotide that comprises a selector codon that is recognized by the O-tRNA, e.g., in vivo or in vitro. In certain embodiments, an O-tRNA of the invention includes at least about, e.g., a 45%, a 50%, a 60%, a 75%, a 80%, or a 90% or more suppression efficiency in the presence of a cognate synthetase in response to a selector codon as compared to an O-tRNA comprising or encoded by a polynucleotide sequence as set forth in the O-tRNA sequences in the sequence listing herein.

Examples of O-tRNAs of the invention are set forth in the sequence listing herein, for example, see the sequence listing. The disclosure herein also provides guidance for the design of additional equivalent O-tRNA species. In an RNA molecule, such as an O-RS mRNA, or O-tRNA molecule, Thymine (T) is replaced with Uracil (U) relative to a given sequence (or vice versa for a coding DNA), or complement thereof. Additional routine modifications to the bases can also be present.

The invention also encompasses conservative variations of O-tRNAs corresponding to particular O-tRNAs herein. For example, conservative variations of O-tRNA include those molecules that function like the particular O-tRNAs, e.g., as in the sequence listing herein and that maintain the tRNA L-shaped structure by virtue of appropriate self-complementarity, but that do not have a sequence identical to that, e.g., in the sequence listing, and desirably, are other than wild type tRNA molecules.

The composition comprising an O-tRNA can further include an orthogonal aminoacyl-tRNA synthetase (O-RS), where the O-RS preferentially aminoacylates the O-tRNA with an unnatural amino acid. In certain embodiments, a composition including an O-tRNA can further include a translation system, e.g., in vitro or in vivo. A nucleic acid that comprises a polynucleotide that encodes a polypeptide of interest, where the polynucleotide comprises a selector codon that is recognized by the O-tRNA, or a combination of one or more of these can also be present in the cell.

Methods for producing a recombinant orthogonal tRNA and screening its efficiency with respect to incorporating an unnatural amino acid into a polypeptide in response to a selector codon can be found, e.g., in International Application Publications WO 2002/086075, entitled “METHODS AND COMPOSITIONS FOR THE PRODUCTION OF ORTHOGONAL tRNA AMINOACYL-tRNA SYNTHETASE PAIRS;” WO 2004/094593, entitled “EXPANDING THE EUKARYOTIC GENETIC CODE;” and WO 2005/019415, filed Jul. 7, 2004. See also Forster, et al., (2003) “Programming peptidomimetic synthetases by translating genetic codes designed de novo.” Proc Natl Acad Sci USA 100: 6353-6357; and Feng, et al., (2003) “Expanding tRNA recognition of a tRNA synthetase by a single amino acid change.” Proc Natl Acad Sci USA 100: 5676-5681. Additional details are found in U.S. Pat. No. 7,045,337; No. 7,083,970; No. 7,238,510; No. 7,129,333; No. 7,262,040; No. 7,183,082; No. 7,199,222; and No. 7,217,809.

Orthogonal Aminoacyl-tRNA Synthetase (O-RS)

The O-RS of the invention preferentially aminoacylates an O-tRNA with an unnatural amino acid, e.g., an aliphatic, aryl or heterocycle substituted boronic acid, e.g., p-boronophenylalanine, o-boronophenylalanine, or m-boronophenylalanine, in vitro or in vivo. The O-RS of the invention can be provided to the translation system, e.g., a bacterial or eukaryotic cell, by a polypeptide that includes an O-RS and/or by a polynucleotide that encodes an O-RS or a portion thereof. For example, an example O-RS comprises an amino acid sequence as set forth in the sequence listing, or a conservative variation thereof. In another example, an O-RS, or a portion thereof, is encoded by a polynucleotide sequence that encodes an amino acid comprising sequence in the sequence listing or examples herein, or a complementary polynucleotide sequence thereof.

General details for producing an O-RS, assaying its aminoacylation efficiency, and/or altering its substrate specificity can be found in Internal Publication Number WO 2002/086075, entitled “METHODS AND COMPOSITIONS FOR THE PRODUCTION OF ORTHOGONAL tRNA AMINOACYL-tRNA SYNTHETASE PAIRS;” and WO 2004/094593, entitled “EXPANDING THE EUKARYOTIC GENETIC CODE.” See also, Wang and Schultz “Expanding the Genetic Code,” Angewandte Chemie Int Ed 44: 34-66 (2005); and Hoben and Soll (1985) Methods Enzymol 113: 55-59, the contents of which are incorporated by reference in their entirety. Additional details are found in U.S. Pat. No. 7,045,337; No. 7,083,970; No. 7,238,510; No. 7,129,333; No. 7,262,040; No. 7,183,082; No. 7,199,222; and No. 7,217,809.

Source and Host Organisms

The orthogonal translational components (O-tRNA and O-RS) of the invention can be derived from any organism, or a combination of organisms, for use in a host translation system from any other species, with the caveat that the O-tRNA/O-RS components and the host system work in an orthogonal manner. It is not a requirement that the O-tRNA and the O-RS from an orthogonal pair be derived from the same organism. In some aspects, the orthogonal components are derived from archaebacterial genes for use in a eubacterial host system.

For example, the orthogonal O-tRNA can be derived from an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei (Mm), Pyrobaculum aerophilum, Pyrococcus abyssi, Sulfolobus solfataricus (Ss), Sulfolobus tokodaii, Thermoplasma acidophilum, Thermoplasma volcanium, or the like, or a eubacterium, such as Escherichia coli, Thermus thermophilus, Bacillus subtilis, Bacillus stearothermphilus, or the like, while the orthogonal O-RS can be derived from an organism or combination of organisms, e.g., an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei, Pyrobaculum aerophilum, Pyrococcus abyssi, Sulfolobus solfataricus, Sulfolobus tokodaii, Thermoplasma acidophilum, Thermoplasma volcanium, or the like, or a eubacterium, such as Escherichia coli, Thermus thermophilus, Bacillus subtilis, Bacillus stearothermphilus, or the like. In one embodiment, eukaryotic sources, e.g., plants, algae, protists, fungi, yeasts, animals, e.g., mammals, insects, arthropods, or the like can also be used as sources of O-tRNAs and O-RSs.

The individual components of an O-tRNA/O-RS pair can be derived from the same organism or different organisms. In one embodiment, the O-tRNA/O-RS pair is from the same organism. Alternatively, the O-tRNA and the O-RS of the O-tRNA/O-RS pair are from different organisms.

The O-tRNA, O-RS or O-tRNA/O-RS pair can be selected or screened in vivo or in vitro and/or used in a cell, e.g., a eubacterial cell, to produce a polypeptide with an unnatural amino acid. The eubacterial cell used is not limited, for example, Escherichia coli, Thermus thermophilus, Bacillus subtilis, Bacillus stearothermphilus, or the like. Compositions of eubacterial cells comprising translational components of the invention are also a feature of the invention.

See also, International Application Publication Number WO 2004/094593, entitled “EXPANDING THE EUKARYOTIC GENETIC CODE,” filed Apr. 16, 2004, for screening O-tRNA and/or O-RS in one species for use in another species. Additional details are found in Wang and Schultz, (2005) “Expanding the Genetic Code.” Angewandte Chemie Int Ed 44: 34-66; Xie and Schultz, (2005) “An Expanding Genetic Code.” Methods 36: 227-238; Xie and Schultz, (2005) “Adding Amino Acids to the Genetic Repertoire.” Curr Opinion in Chemical Biology 9: 548-554; and Wang, et al., (2006) “Expanding the Genetic Code.” Annu Rev Biophys Biomol Struct 35: 225-249, and U.S. Pat. No. 7,045,337; No. 7,083,970; No. 7,238,510; No. 7,129,333; No. 7,262,040; No. 7,183,082; No. 7,199,222; and No. 7,217,809.

Selector Codons

Selector codons of the invention expand the genetic codon framework of protein biosynthetic machinery. For example, a selector codon includes, e.g., a unique three base codon, a nonsense codon, such as a stop codon, e.g., an amber codon (UAG), or an opal codon (UGA), an unnatural codon, at least a four base codon, a rare codon, or the like. A number of selector codons can be introduced into a desired gene, e.g., one or more, two or more, more than three, etc. Conventional site-directed mutagenesis can be used to introduce the selector codon at the site of interest in a polynucleotide encoding a polypeptide of interest. See, e.g., Sayers, J. R., et al. (1988) “5′, 3′ Exonuclease in phosphorothioate-based oligonucleotide-directed mutagenesis.” Nucl Acid Res 16: 791-802. By using different selector codons, multiple orthogonal tRNA/synthetase pairs can be used that allow the simultaneous site-specific incorporation of multiple unnatural amino acids e.g., including at least one unnatural amino acid, using these different selector codons.

Unnatural amino acids can also be encoded with rare codons. For example, when the arginine concentration in an in vitro protein synthesis reaction is reduced, the rare arginine codon, AGG, has proven to be efficient for insertion of Ala by a synthetic tRNA acylated with alanine. See, e.g., Ma, C. et al., (1993) “In vitro protein engineering using synthetic tRNA^{Ala} with different anticodons.” Biochemistry 32: 7939-7945. In this case, the synthetic tRNA competes with the naturally occurring tRNA^{Arg}, which exists as a minor species in Escherichia coli. In addition, some organisms do not use all triplet codons. An unassigned codon AGA in Micrococcus luteus has been utilized for insertion of amino acids in an in vitro transcription/translation extract. See, e.g., Kowal and Oliver, (1997) “Exploiting unassigned codons in Micrococcus luteus for tRNA-based amino acid mutagenesis.” Nucl Acid Res 25: 4685-4689. Components of the invention can be generated to use these rare codons in vivo.

Selector codons can also comprise extended codons, e.g., four or more base codons, such as, four, five, six or more base codons. Examples of four base codons include, e.g., AGGA, CUAG, UAGA, CCCU, and the like. Examples of five base codons include, e.g., AGGAC, CCCCU, CCCUC, CUAGA, CUACU, UAGGC and the like. Methods of the invention include using extended codons based on frameshift suppression. Four or more base codons can insert, e.g., one or multiple unnatural amino acids, into the same protein. In other embodiments, the anticodon loops can decode, e.g., at least a four-base codon, at least a five-base codon, or at least a six-base codon or more. Since there are 256 possible four-base codons, multiple unnatural amino acids can be encoded in the same cell using a four or more base codon. See also, Anderson, et al., (2002) “Exploring the Limits of Codon and Anticodon Size.” Chemistry and Biology 9: 237-244; Magliery, et al., (2001) “Expanding the Genetic Code: Selection of Efficient Suppressors of Four-base Codons and Identification of “Shifty” Four-base Codons with a Library Approach in Escherichia coli.” J Mol Biol 307: 755-769; Ma, C., et al., (1993) “In vitro protein engineering using synthetic tRNA^{Ala} with different anticodons.” Biochemistry 32:7939; Hohsaka, et al., (1999) “Efficient Incorporation of Non-natural Amino Acids with Large Aromatic Groups into Streptavidin in In Vitro Protein Synthesizing Systems.” J Am Chem Soc 121: 34-40; and Moore, et al., (2000) “Quadruplet Codons: Implications for Code Expansion and the Specification of Translation Step Size.” J Mol Biol 298: 195-209. Four base codons have been used as selector codons in a variety of orthogonal systems. See, e.g., WO 2005/019415; WO 2005/007870 and WO 2005/07624. See also, Wang and Schultz, (2005) “Expanding the Genetic Code.” Angewandte Chemie Int Ed 44: 34-66, the content of which is incorporated by reference in its entirety.

For a given system, a selector codon can also include one of the natural three base codons, where the endogenous system does not use (or rarely uses) the natural base codon. For example, this includes a system that is lacking a tRNA that recognizes the natural three base codon, and/or a system where the three base codon is a rare codon.

Selector codons optionally include unnatural base pairs. Descriptions of unnatural base pairs which can be adapted for methods and compositions include, e.g., Hirao, et al., (2002) “An unnatural base pair for incorporating amino acid analogues into protein.” Nature Biotechnology 20: 177-182. See also Wu, et al., (2002) “Enzymatic Phosphorylation of Unnatural Nucleosides.” J Am Chem Soc 124: 14626-14630.

### Nucleic Acid and Polypeptide Sequences and Variants

As described herein, the invention provides for polynucleotide sequences encoding, e.g., O-tRNAs and O-RSs, and polypeptide amino acid sequences, e.g., O-RSs, and, e.g., compositions, systems and methods comprising said polynucleotide or polypeptide sequences. Examples of said sequences, e.g., O-tRNA and O-RS amino acid and nucleotide sequences are disclosed herein (see the sequence listing). However, one of skill in the art will appreciate that the invention is not limited to those sequences disclosed herein, e.g., in the Examples and sequence listing. One of skill will appreciate that the invention also provides many related sequences with the functions described herein, e.g., polynucleotides and polypeptides encoding conservative variants of an O-RS disclosed herein.

As used herein, the term “conservative variant,” in the context of a translation component, refers to a translation component, e.g., a conservative variant O-tRNA or a conservative variant O-RS, that functionally performs similar to a base component that the conservative variant is similar to, e.g., an O-tRNA or O-RS, having variations in the sequence as compared to a reference O-tRNA or O-RS. For example, an O-RS, or a conservative variant of that O-RS, will aminoacylate a cognate O-tRNA with p-boronophenylalanine. In this example, the O-RS and the conservative variant O-RS do not have the same amino acid sequences. The conservative variant can have, e.g., one variation, two variations, three variations, four variations, or five or more variations in sequence, as long as the conservative variant is still complementary to, e.g., functions with, the cognate corresponding O-tRNA or O-RS.

In some embodiments, a conservative variant O-RS comprises one or more conservative amino acid substitutions compared to the O-RS from which it was derived. In some embodiments, a conservative variant O-RS comprises one or more conservative amino acid substitutions compared to the O-RS from which it was derived, and furthermore, retains O-RS biological activity; for example, a conservative variant O-RS that retains at least 10% of the biological activity of the parent O-RS molecule from which it was derived, or alternatively, at least 20%, at least 30%, or at least 40%. In some preferred embodiments, the conservative variant O-RS retains at least 50% of the biological activity of the parent O-RS molecule from which it was derived. The conservative amino acid substitutions of a conservative variant O-RS can occur in any domain of the O-RS, including the amino acid binding pocket.

Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the polypeptide molecule. The following sets forth example groups that contain natural amino acids of like chemical properties, where substitutions within a group is a “conservative substitution”.

TABLE A
Conservative Amino Acid Substitutions

| | | | Positively | Negatively |
|---|---|---|---|---|
| Nonpolar and/or | Polar, | Aromatic | Charged | Charged |
| Aliphatic Side | Uncharged | Side | Side | Side |
| Chains | Side Chains | Chains | Chains | Chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

In one aspect, conservative substitutions of an RS sequence listed in the sequence listing will retain a Ser or Gly residue at position 32, an alanine at position 65, a His or Met residue at position 70, a Ser or Ala residue at position 158, a glutamine at position 162 or a combination thereof, where amino acid position numbering corresponds to amino acid position numbering of the wild-type tyrosyl tRNA synthetase.

Owing to the degeneracy of the genetic code, “silent substitutions”, i.e., substitutions in a nucleic acid sequence which do not result in an alteration in an encoded polypeptide, are an implied feature of every nucleic acid sequence that encodes an amino acid sequence. Similarly, “conservative amino acid substitutions,” where one or a limited number of amino acids in an amino acid sequence are substituted with different amino acids with highly similar properties, are also readily identified as being highly similar to a disclosed construct. Such conservative variations of each disclosed sequence are a feature of the present invention.

In one aspect, the invention can include O-tRNAs and O-RS that are “derived from” a parental molecule. As used herein, the term “derived from” refers to a component that is isolated from or made using a specified molecule or organism, or information from the specified molecule or organism. For example, a polypeptide that is derived from a second polypeptide can include an amino acid sequence that is identical or substantially similar to the amino acid sequence of the second polypeptide. In the case of polypeptides, the derived species can be obtained by, for example, naturally occurring mutagenesis, artificial directed mutagenesis or artificial random mutagenesis. The mutagenesis used to derive polypeptides can be intentionally directed or intentionally random, or a mixture of each. The mutagenesis of a polypeptide to create a different polypeptide derived from the first can be a random event, e.g., caused by polymerase infidelity, and the identification of the derived polypeptide can be made by appropriate screening methods, e.g., as discussed herein. Mutagenesis of a polypeptide typically entails manipulation of the polynucleotide that encodes the polypeptide.

As used herein, one type of biomolecule can “encode” another. As used herein, the term “encode” refers to any process whereby the information in a polymeric macromolecule or sequence string is used to direct the production of a second molecule or sequence string that is different from the first molecule or sequence string. As used herein, the term can be used broadly, and can have a variety of applications. In some aspects, the term “encode” describes the process of semi-conservative DNA replication, where one strand of a double-stranded DNA molecule is used as a template to encode a newly synthesized complementary sister strand by a DNA-dependent DNA polymerase. In another aspect, the term “encode” refers to any process whereby the information in one molecule is used to direct the production of a second molecule that has a different chemical nature from the first molecule. For example, a DNA molecule can encode an RNA molecule, e.g., by the process of transcription incorporating a DNA-dependent RNA polymerase enzyme. Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

Nucleic Acid Hybridization

Comparative hybridization can also be used to identify nucleic acids of the invention, including conservative variations of nucleic acids of the invention. In addition, target nucleic acids which hybridize to a nucleic acid represented in the sequence listing herein, under high, ultra-high and ultra-ultra high stringency conditions, where the nucleic acids encode mutations corresponding to: a Ser or Gly residue at position 32, an alanine at position 65, a His or Met residue at position 70, a Ser or Ala residue at position 158, a glutamine at position 162 or a combination thereof, with amino acid position numbering corresponding to amino acid position numbering of the wild-type tyrosyl tRNA synthetase.

Examples of such nucleic acids include those with one or a few silent or conservative nucleic acid substitutions as compared to a given nucleic acid sequence of the sequence listing, e.g., which also include, e.g., a Ser or Gly residue at position 32, an alanine at position 65, a His or Met residue at position 70, a Ser or Ala residue at position 158, a glutamine at position 162 or a combination thereof, wherein amino acid position numbering corresponds to amino acid position numbering of the wild-type tyrosyl tRNA synthetase.

A test nucleic acid is said to specifically hybridize to a probe nucleic acid when it hybridizes at least 50% as well to the probe as to the perfectly matched complementary target, i.e., with a signal to noise ratio at least half as high as hybridization of the probe to the target under conditions in which the perfectly matched probe binds to the perfectly matched complementary target with a signal to noise ratio that is at least about 5×-10× as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Current Protocols in Molecular Biology, Ausubel, et al., eds., Current Protocols, a joint venture between Greene Publishing Associates, Inc. and John Wiley & Sons, Inc., (supplemented through 2004) (“Ausubel”); Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions is a 0.2×SSC wash at 65° C. for 15 minutes (see, Sambrook, supra for a description of SSC buffer). Often the high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assay indicates detection of a specific hybridization.

“Stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993), supra. and in Hames and Higgins, 1 and 2. Stringent hybridization and wash conditions can easily be determined empirically for any test nucleic acid. For example, in determining stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, in highly stringent hybridization and wash conditions, the hybridization and wash conditions are gradually increased until a probe binds to a perfectly matched complementary target with a signal to noise ratio that is at least 5× as high as that observed for hybridization of the probe to an unmatched target.

“Very stringent” conditions are selected to be equal to the thermal melting point (Tₘ) for a particular probe. The Tₘ is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tₘ for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

### Additional Details Regarding Techniques

Additional useful references for producing RS and tRNA mutations, as well as a variety of recombinant and in vitro nucleic acid manipulation methods (including cloning, expression, PCR, and the like) include Berger and Kimmel, Guide to Molecular Cloning Techniques, Methods in Enzymology volume 152 Academic Press, Inc., San Diego, Calif. (Berger); Kaufman, et al. (2003) Handbook of Molecular and Cellular Methods in Biology and Medicine Second Edition Ceske (ed) CRC Press (Kaufman); and The Nucleic Acid Protocols Handbook Ralph Rapley (ed) (2000) Cold Spring Harbor, Humana Press Inc (Rapley); Chen, et al. (ed) PCR Cloning Protocols, Second Edition (Methods in Molecular Biology, volume 192) Humana Press; and in Viljoen, et al. (2005) Molecular Diagnostic PCR Handbook Springer, ISBN 1402034032.

A variety of protein methods are known and can be used to isolate, detect, manipulate or otherwise handle a protein produced according to the invention e.g., from recombinant cultures of cells expressing the recombinant borono-containing proteins of the invention. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag, et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000). These available methods are optionally used in conjunction with the novel protein purification methods herein, e.g., scarless protein purification methods.

### Kits

Kits are also a feature of the invention. For example, such kits can comprise components for using the composition herein, such as: a container to hold the kit components, instructional materials for practicing any method herein with the kit, or for producing a protein comprising one or more boronic amino acid, a nucleic acid comprising a polynucleotide sequence encoding an O-tRNA, a nucleic acid comprising a polynucleotide encoding an O-RS, an O-RS, a boronic amino acid, reagents for the post-translational modification of the unnatural amino acid (e.g., reagents for any one or more of the reactions described herein), a suitable strain of prokaryotic, e.g., bacterial (e.g., E. coli) or eukaryotic (e.g., yeast or mammalian) host cells for expression of the O-tRNA/O-RS and production of a target protein comprising, e.g., one or more an aliphatic, aryl or heterocycle substituted boronic acid, p-boronophenylalanine, m-boronophenylalanine and/or o-boronophenylalanine.

Alternately or additionally, the kits can contain a solid phase matrix for scarless purification, reagents for the covalent coupling of a polypeptide comprising a boronic amino acid to the matrix, and/or reagents for the oxidation or reduction of the boronic amino acid in a polypeptide to produce a natural amino acid.

### ADDITIONAL DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

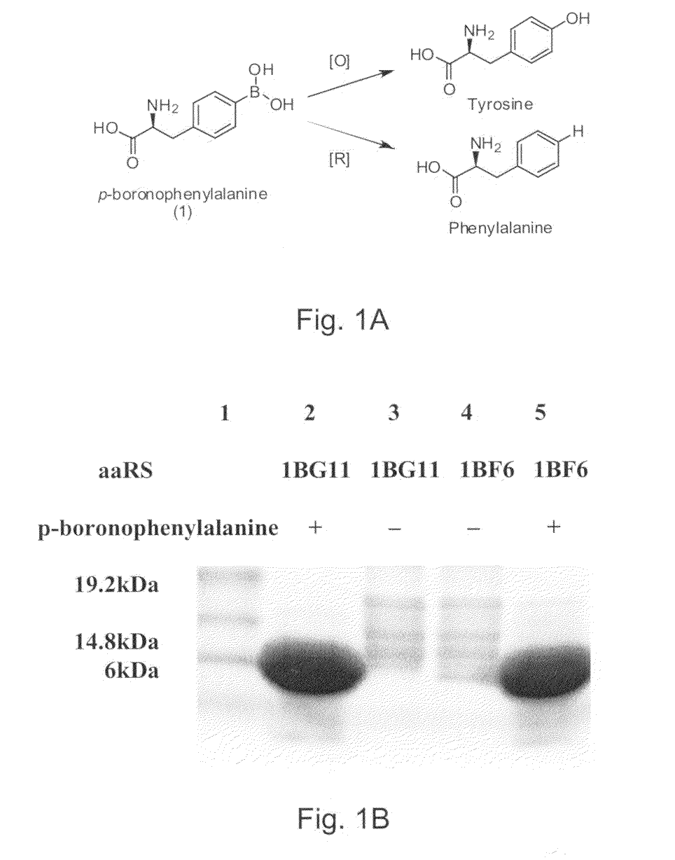

A boronic amino acid (also referred to as a “borono amino acid”) is an amino acid that comprises a boron moiety. For example, p-boronophenylalanine is described in FIG. 1A. p-boronophenylalanine is also known as dihydroxyborylphenylalanine and as p-boronylphenylalanine. m and o-boronophenylalanine, as well as aliphatic, aryl and heterocycle substituted boronic amino acids are described herein.

Orthogonal: As used herein, the term “orthogonal” refers to functional molecules, e.g., an orthogonal tRNA (O-tRNA) and/or an orthogonal aminoacyl-tRNA synthetase (O-RS), that function poorly or not at all with endogenous components of a cell, when compared to a corresponding molecule (tRNA or RS) that is endogenous to the cell or translation system. Orthogonal components are usefully provided as cognate components that function well with each other, e.g., an O-RS can be provided that can efficiently aminoacylates a cognate O-tRNA in a cell, even though the O-tRNA functions poorly or not at all as a substrate for the endogenous RS of the cell, and the O-RS functions poorly or not at all with endogenous tRNAs of the cell. Various comparative efficiencies of the orthogonal and endogenous components can be evaluated. For example, an O-tRNA will typically display poor or non-existent activity as a substrate, under typical physiological conditions, with endogenous RSs, e.g., the O-tRNA is less than 10% as efficient as a substrate as endogenous tRNAs for any endogenous RS, and will typically be less than 5%, and usually less than 1% as efficient a substrate. At the same time, the tRNA can be highly efficient as a substrate for the O-RS, e.g., at least 50%, and often 75%, 95%, or even 100% or more as efficient as an aminoacylation substrate as any endogenous tRNA is for its endogenous RS.

Orthogonal aminoacyl-tRNA synthetase: As used herein, an orthogonal aminoacyl-tRNA synthetase (O-RS) is an enzyme that preferentially aminoacylates an O-tRNA with an amino acid in a translation system of interest. The amino acid that the O-RS loads onto the O-tRNA in the present invention is a boronic amino acid, e.g., an aliphatic, aryl or heterocycle substituted boronic amino acid, e.g., a p, m or o-boronophenylalanine. An ORS “selectively recognizes” an unnatural amino acid when it charges a cognate tRNA with the amino acid more efficiently than with any natural amino acid.

Orthogonal tRNA: As used herein, an orthogonal tRNA (O-tRNA) is a tRNA that is orthogonal to a translation system of interest. The O-tRNA can exist charged with, e.g., a boronic amino acid, or can exist in an uncharged state. It is also to be understood that an O-tRNA is optionally charged (aminoacylated) by a cognate orthogonal aminoacyl-tRNA synthetase with a boronic amino acid. Indeed, it will be appreciated that the O-tRNA of the invention is most advantageously used to insert the boronic amino acid into a growing polypeptide, during translation, in response to a selector codon.

Preferentially aminoacylates: As used herein in reference to orthogonal translation systems, an O-RS “preferentially aminoacylates” a cognate O-tRNA when the O-RS charges the O-tRNA with p-boronophenylalanine more efficiently than it charges any endogenous tRNA in an expression system. That is, when the O-tRNA and any given endogenous tRNA are present in a translation system in approximately equal molar ratios, the O-RS will charge the O-tRNA more frequently than it will charge the endogenous tRNA. Preferably, the relative ratio of O-tRNA charged by the O-RS to endogenous tRNA charged by the O-RS is high, preferably resulting in the O-RS charging the O-tRNA exclusively, or nearly exclusively, when the O-tRNA and endogenous tRNA are present in equal molar concentrations in the translation system. The relative ratio between O-tRNA and endogenous tRNA that is charged by the O-RS, when the O-tRNA and O-RS are present at equal molar concentrations, is greater than 1:1, preferably at least about 2:1, more preferably 5:1, still more preferably 10:1, yet more preferably 20:1, still more preferably 50:1, yet more preferably 75:1, still more preferably 95:1, 98:1, 99:1, 100:1, 500:1, 1,000:1, 5,000:1 or higher. The O-RS “preferentially aminoacylates an O-tRNA with a boronic amino acid” when (a) the O-RS preferentially aminoacylates the O-tRNA compared to an endogenous tRNA, and (b) where that aminoacylation is specific for the boronic amino acid, as compared to aminoacylation of the O-tRNA by the O-RS with any natural amino acid. For example, when a p-boronophenylalanine and natural amino acids are present in equal molar amounts in a translation system comprising a relevant O-RS of the sequence listing herein and a relevant O-tRNA of the sequence listing herein, the O-RS will load the O-tRNA with p-boronophenylalanine more frequently than with any natural amino acid. Preferably, the relative ratio of O-tRNA charged with p-boronophenylalanine to O-tRNA charged with the natural amino acid is high. More preferably, O-RS charges the O-tRNA exclusively, or nearly exclusively, with the p-boronophenylalanine or other relevant borono amino acid. The relative ratio between charging of the O-tRNA with the boronic amino acid and charging of the O-tRNA with a natural amino acid, when both the natural and boronic amino acid are present in the translation system in equal molar concentrations, is greater than 1:1, preferably at least about 2:1, more preferably 5:1, still more preferably 10:1, yet more preferably 20:1, still more preferably 50:1, yet more preferably 75:1, still more preferably 95:1, 98:1, 99:1, 100:1, 500:1, 1,000:1, 5,000:1 or higher.

Selector codon: The term “selector codon” refers to codons recognized by the O-tRNA in the translation process and not recognized by an endogenous tRNA. The O-tRNA anticodon loop recognizes the selector codon on the mRNA and incorporates the amino acid with which it is charged, e.g., p-boronophenylalanine, at this site in the polypeptide. Selector codons can include, e.g., nonsense codons, such as, stop codons, e.g., amber, ochre, and opal codons; four or more base codons; rare codons; noncoding codons; and codons derived from natural or unnatural base pairs and/or the like.

Suppression activity: As used herein, the term “suppression activity” refers, in general, to the ability of a tRNA, e.g., a suppressor tRNA, to allow translational read-through of a codon, e.g., a selector codon that is an amber codon or a 4- or -more base codon, that would otherwise result in the termination of translation or mistranslation, e.g., frame-shifting. Suppression activity of a suppressor tRNA can be expressed as a percentage of translational read-through activity observed compared to a second suppressor tRNA, or as compared to a control system, e.g., a control system lacking an O-RS.

Suppressor tRNA: A suppressor tRNA is a tRNA that alters the reading of a messenger RNA (mRNA) in a given translation system, typically by allowing the incorporation of an amino acid in response to a stop codon (i.e., “read-through”) during the translation of a polypeptide. In some aspects, a selector codon of the invention is a suppressor codon, e.g., a stop codon, e.g., an amber, ocher or opal codon, a four base codon, a rare codon, etc.

A therapeutic protein is a protein that can be administered to a patient to treat a disease or disorder.

Translation system: The term “translation system” refers to the components that incorporate an amino acid into a growing polypeptide chain (protein). Components of a translation system can include, e.g., ribosomes, tRNAs, synthetases, mRNA and the like. The O-tRNA and/or the O-RSs of the invention can be added to or be part of an in vitro or in vivo translation system, e.g., in a non-eukaryotic cell, e.g., a bacterium, such as E. coli, or in a eukaryotic cell, e.g., a yeast cell, a mammalian cell, a plant cell, an algae cell, a fungus cell, an insect cell, and/or the like.

Unnatural amino acid: As used herein, the term “unnatural amino acid” refers to any amino acid, modified amino acid, and/or amino acid analogue, that is not one of the 20 common naturally occurring amino acids. For example, the unnatural amino acid p-boronophenylalanine (see FIG. 1A) finds use with the invention.

### EXAMPLES

The following examples are offered to illustrate, but not to limit the claimed invention. One of skill will immediately recognize a variety of non-critical parameters that can be modified to achieve essentially similar results.

It is understood that the examples and embodiments described herein are for illustrative purposes only and that various modifications or changes in light thereof will be suggested to persons skilled in the art and are to be included within the spirit and purview of this application and scope of the appended claims.

### Example 1

### A Genetically Encoded Boronate Amino Acid

In this example, we describe a general methodology for the site-specific incorporation of p-boronophenylalanine (FIG. 1A, Compound 1) into proteins directly in E. coli. We also demonstrate the utility of this system for boronate mediated protein modification as well as the single step scar-less purification of proteins.

We have previously described methodology for the addition of unnatural amino acids to the genetic code of E. coli (20), yeast (21), and mammalian cells (22). This method is based on the generation of an orthogonal tRNA/aminoacyl-tRNA synthetase (aaRS) pair that allows the site-selective incorporation of a novel amino acid into proteins in response to unique nonsense and frameshift codons. To selectively incorporate p-boronophenylalanine into proteins in E. coli, we have used a Methanococcus jannaschii (Mj) derived amber suppressor tyrosyl TRNA (MjtRNA^{Tyr}_{CUA})/tyrosyl-tRNA synthetase (MjTyrRS) pair that has been previously shown to function efficiently in E. coli, but does not cross react with any of the endogenous tRNAs and aminoacyl tRNA synthetases (23, 24). To alter the specificity of the MjTyrRS synthetase to selectively recognize p-boronophenylalanine, two active site NNK saturation mutagenesis libraries of >10⁹ diversity were each subjected to iterative rounds of positive and negative selections. The design of these libraries and the selection methodology has been described elsewhere (20, 25). Briefly, for positive selections, survival in the presence of 1 mM boronate amino acid is contingent on the suppression of an amber mutation in the chloramphenicol acetyl transferase gene (CAT), which confers resistance to the antibiotic chloramphenicol. Surviving clones are then subjected to a round of negative selection in the absence of the unnatural amino acid in which suppression of three amber mutations in the toxic barnase gene selectively removes aaRS variants that incorporate any of the 20 canonical amino acids.

After selections (3 positive and 2 negative rounds) numerous clones were obtained that permitted cells harboring the CAT selection plasmid to survive on 120 μg/mL of chloramphenicol only in presence of 1 mM p-boronophenylalanine. Absence of the amino acid precluded cell growth in this system. The most active aaRS variants from this selection came from a single aminoacyl-tRNA synthetase library comprised of randomized active site mutations at codons Tyr32, Leu65, His70, Gln155, Asp158, and Leu162. As shown in Table 1, of the seven clones sequenced, three unique sequences were obtained displaying considerable consensus. Positions 65 and 162 showed complete convergence to alanine and glutamate, respectively, while position 155 maintained the wildtype glutamine for all clones. Positions 32, 70, and 158, were enriched for Ser/Gly, His/Met, or Ser/Ala, respectively. Analysis of the wild type MjTyrRS crystal structure complexed with tyrosine provides some rationale for the possible roles of these mutations (26). Tyr32 and Asp158 make critical hydrogen bonds to the phenolic oxygen of the bound tyrosine. Replacement of these amino acids with a smaller serine residue removes the determinants necessary for binding to tyrosine, while maintaining hydrogen bonding functionality that may interact with the boronate group. Mutation of Leu162 to Asp adds an additional hydrogen bonding residue that could interact with the boronate functionality. The most common sequence (1BG11, designated B(OH)₂PheRS) was chosen for characterization and used for all subsequent experiments.

TABLE 1
Mutations for evolved p-boronophenylalanine synthetases
obtained from selections.

Position

| Clone | 32 | 65 | 70 | 155 | 158 | 162 |
|---|---|---|---|---|---|---|
| WT | Tyr | Leu | His | Gln | Asp | Leu |
| 1BF6 | Ser | Ala | His | Gln | Ser | Glu |
| 1BF9 | Ser | Ala | His | Gln | Ser | Glu |
| 1BE3 | Gly | Ala | His | Gln | Ala | Glu |
| 1BF10 | Ser | Ala | Met | Gln | Ser | Glu |
| 1BF12 | Ser | Ala | Met | Gln | Ser | Glu |
| 1BG10 | Ser | Ala | Met | Gln | Ser | Glu |
| 1BG11 | Ser | Ala | Met | Gln | Ser | Glu |

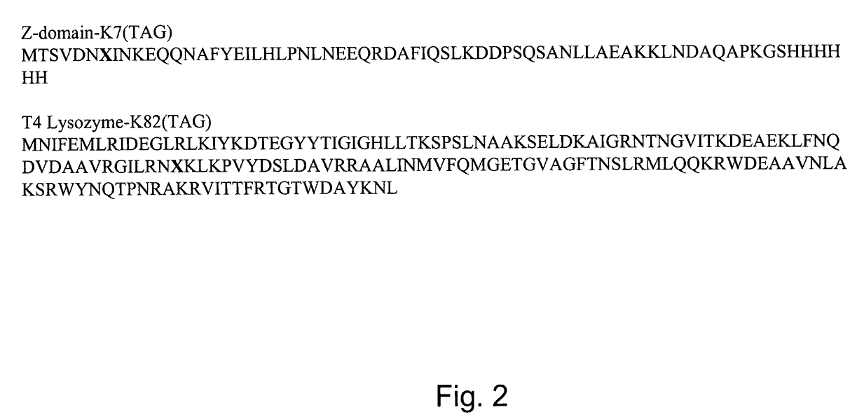

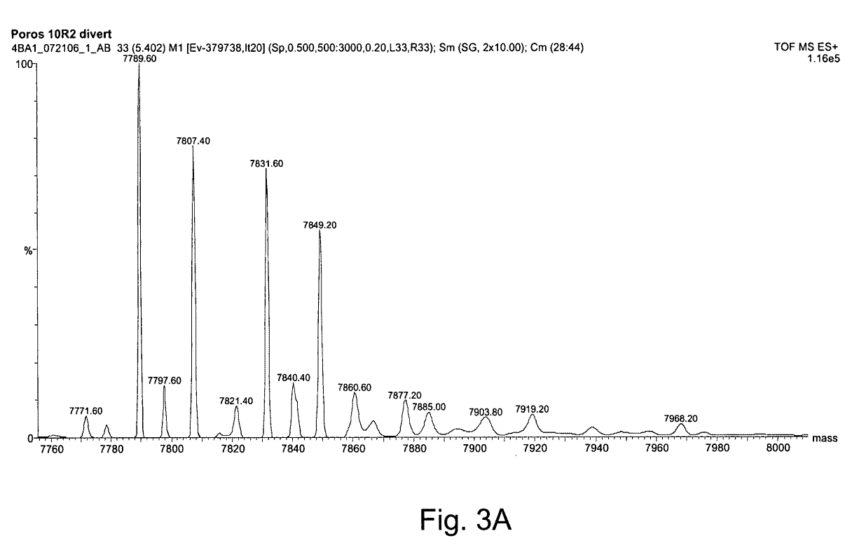

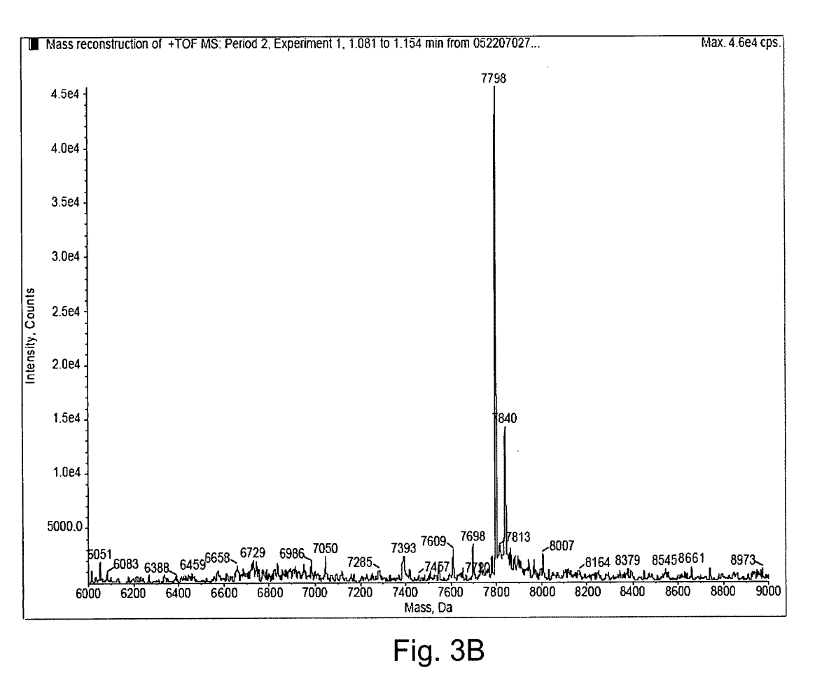

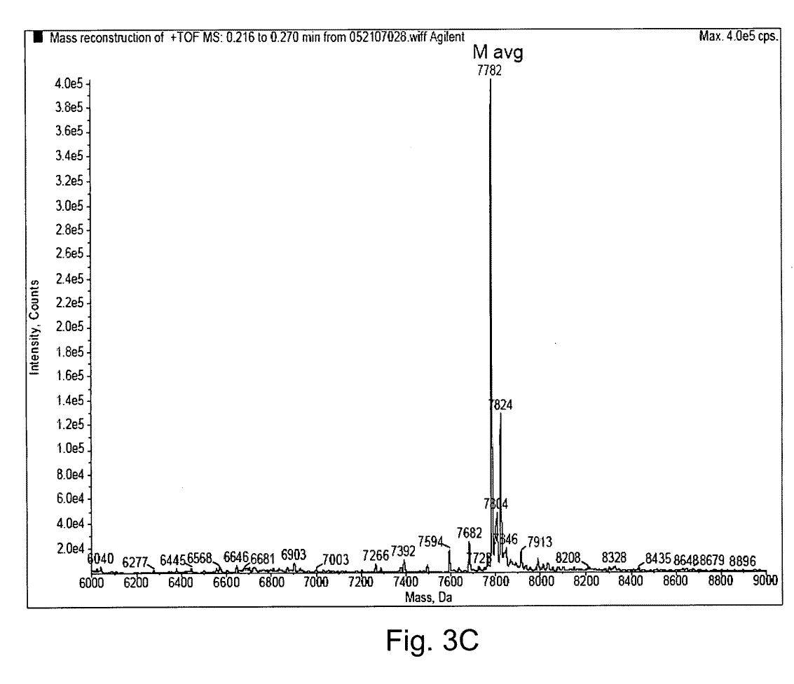

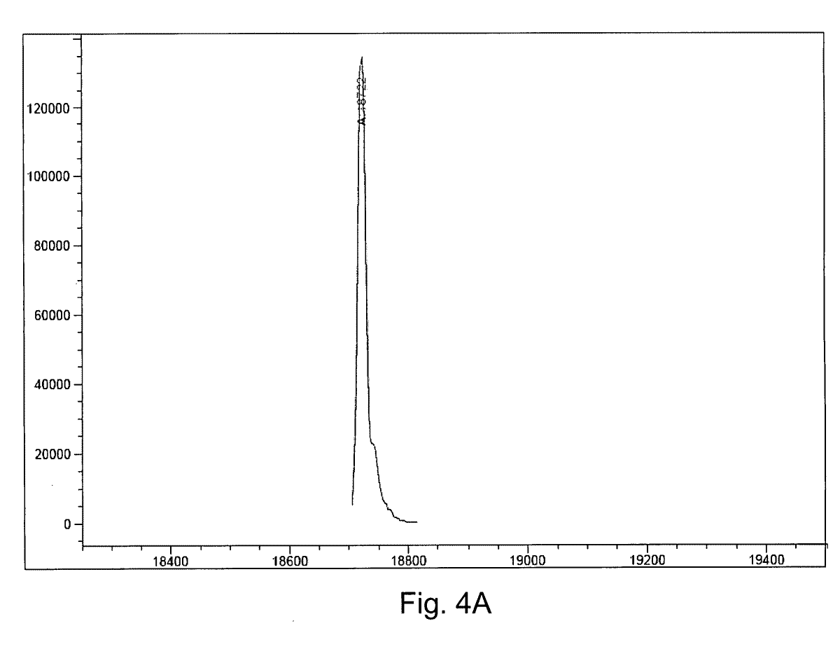

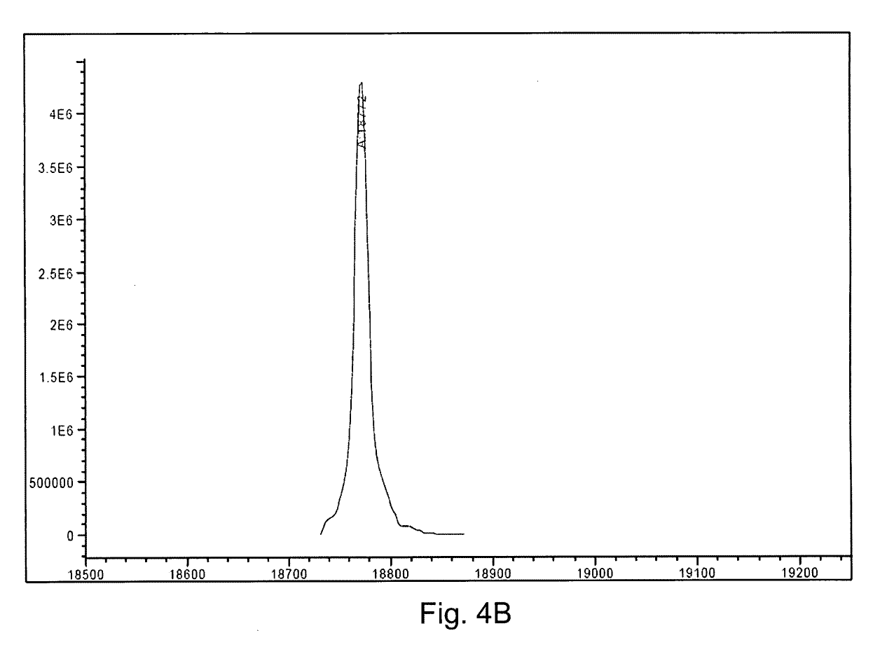

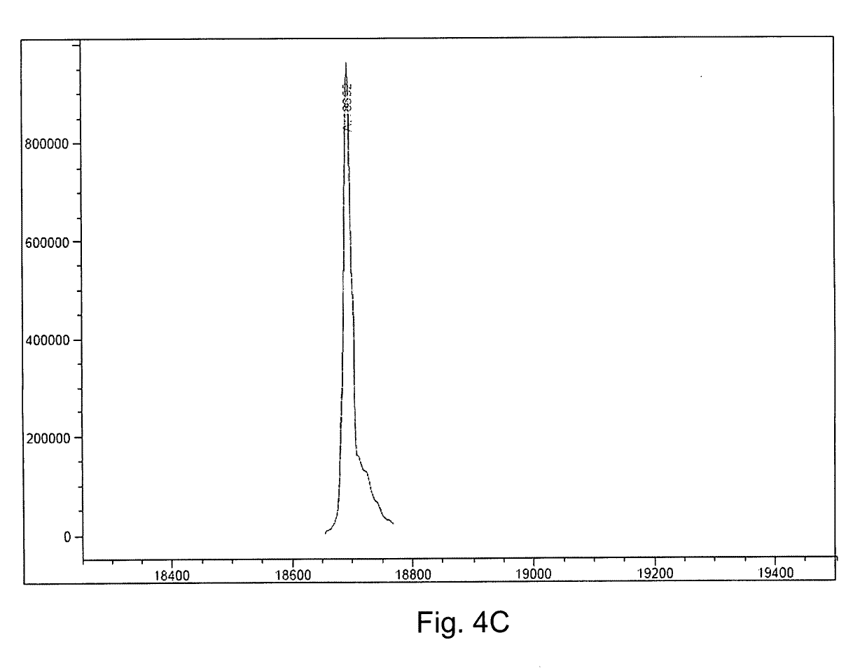

To confirm the incorporation of p-boronophenylalanine into proteins, an amber mutation (TAG) was substituted for Lys7 of a C-terminal 6×His tagged variant of the Z-domain of staphylococcal protein A (FIG. 2). FIG. 2 depicts protein sequences for Z-domain and T4 lysozyme mutants, and X indicates the position of the unnatural amino acid as encoded by the amber (TAG) stop codon. Expression experiments were carried out in DH10B E. coli cells harboring plasmids containing the amber Z-domain gene as well as the MjtRNA^{Tyr}_{CUA} and the evolved B(OH)₂PheRS. Protein was expressed in 2×YT media in the presence or absence of 1 mM p-boronophenylalanine followed by nickel affinity purification. Analysis by SDS-PAGE and subsequent Coomassie staining showed that protein was only produced in the presence of the boronate amino acid (FIG. 1B). FIG. 1B depicts the results of SDS-PAGE analysis of Z-domain-K7(TAG) protein expression with evolved aaRS, e.g., the B(OH)₂PheRS described above. Z-domain is indicated by the arrow. Lane 1: protein ladder; Lane 2: B(OH)₂PheRS 1BG11+p-boronophenylalanine; Lane 3: B(OH)₂PheRS 1BG11−p-boronophenylalanine; Lane 4: B(OH)₂PheRS 1BF6-p-boronophenylalanine; Lane 5: B(OH)₂PheRS1BF6+p-boronophenylalanine. Protein yields were typically around 15 mg/L of expressed cell culture. The expected mass of the boronate containing Z-domain (Z-domain-K7(p-boronophenylalanine)) is 7824 (M+H protein mass minus the N-terminal methionine which is cleaved post-translationally); however, electrospray ionization mass spectrometry (ESI) of the protein showed 2 peaks corresponding to the loss of 1 or 2 waters (7807 and 7788, respectively, FIG. 3A). FIG. 3A depicts the reconstructed ESI-TOF of Z-domain K7(p-boronophenylalanine). Peaks at 7807 (calc. 7807) and 7789 (calc. 7789) correspond to the boronate containing protein minus 1 or 2 waters, respectively. Peaks at 7831 and 7849 correspond to acetylated versions of these two proteins. Acetylation is a common post-translational modification seen with Z-domain constructs expressed in E. coli. Analysis of the Z-domain crystal structure shows that position 7 lies in close proximity to Thr2 and Ser3 on a flexible N-terminal loop (27). These masses are consistent with the formation of boronic esters with the hydroxyl groups of these residues. To further confirm the incorporation p-boronophenylalanine, we substituted Ala82 with p-boronophenylalanine in an engineered cysteine-free T4 lysozyme (T4L-A82(p-boronophenylalanine)). This location was chosen since it is remote to any free hydroxyl groups in the protein structure. ESI of this protein showed the expected mass corresponding to the intact boronate amino acid (calc. 18721, obs. 18722; FIG. 4A) with no dehydration products observed. FIG. 4A depicts the reconstructed ESI of T4L-A82(p-boronophenylalanine). Calc. 18721; Obs. 18722.

p-Boronophenylalanine has been used in solid phase peptide synthesis as a precursor to tyrosine and phenylalanine through the oxidation or reduction of the boronate functionality, respectively (FIGS. 1A and 5A) (28). This reactivity allowed the incorporation of the amino acid to be confirmed by chemical methods. Oxidation of Z-domain-K7(p-boronophenylalanine) for two hours with excess hydrogen peroxide led to the disappearance of both dehydration peaks in the mass spectrum described above to give a single ESI peak corresponding to the expected mass of tyrosine at position 7 (calc. 7798 (M+H), obs. 7798; FIG. 3B). FIG. 3B depicts the reconstructed ESI-TOF of Z-domain-K7(p-boronophenylalanine) after 2 hour incubation with 100 mM hydrogen peroxide. Expected Z-domain-K7Y mass calc. 7798 (M+H); Obs. 7798; Peak at 7840 corresponds to the acetylated protein. Similarly, overnight reduction of Z-domain-K7(p-boronophenylalanine) with excess silver diammonia nitrate yielded the expected mass of the phenylalanine product (calc. 7782 (M+H), obs. 7782; FIG. 3C). FIG. 3C depicts the reconstructed ESI-TOF of Z-domain Z-domain-K7(p-boronophenylalanine) after overnight incubation with 10 mM silver diammonia nitrate. Expected Z-domain-K7F mass calc. 7782 (M+H); Obs. 7782; Peak at 7824 corresponds to the acetylated protein. Using hydrogen peroxide to oxidize the boronate amino acid is not selective in the presence of other easily oxidized residues such as methionine. Indeed, attempts to oxidize T4L-A82(p-boronophenylalanine) (calc. 18693 [M+H]) gave a mass approximately 80 Daltons larger than expected (obs. 18772, FIG. 4B) corresponding to oxidation of each of the 5 methionine residues to methionine sulfoxide. FIG. 4B depicts reconstructed ESI of T4L-A82(p-boronophenylalanine) after oxidation with 100 mM hydrogen peroxide. Expected T4L-A82Y mass calc. 18693 (M+H); Observed: 18772. Observed difference of 79 (˜80) corresponds to the oxidation of each of the five T4L methionines to methionine sulfoxide. Selective oxidation of the boronate moiety was achieved by using one equivalent of potassium peroxymonosulfate (oxone®) (29) which afforded the expected tyrosine product with no observed oxidation of the endogenous methionines (FIG. 4C). FIG. 4C depicts reconstructed ESI of T4L-A82(p-boronophenylalanine) after oxidation with 1 eq. of oxone®. Expected T4L-A82Y mass calc. 18693 (M+H); Observed: 18692.

We next examined the utility of this amino acid as an orthogonal handle to selectively modify proteins. Boronic acids are known to form strong reversible covalent interactions with polyhydroxylated compounds in aqueous solution at physiological pH (6, 30). The interaction of boronates with sorbitol and glucamine (FIG. 5A, Compound 2; R1, R2=H) for example are of very high affinity and provide a useful method to selectively label proteins under conditions that are relatively benign to biological systems. To examine the ability of polyhydroxylated molecules to bind boronate containing proteins, we first synthesized a glucamine functionalized fluorescent reporter. NHS-fluorescein was conjugated to glucamine in DMF quantitatively in one step to afford dye 3, a fluorescein-NHS-glucamine probe (FIG. 5A, Compound 3; R1=NHS-Fluorescein, R2=H) which was then incubated with T4 Lysozyme containing the boronate amino acid at position 82 (or tyrosine at the same location as a negative control) in 50 mM CHES buffer, pH 8.5. After 1 hr at room temperature, excess dye was removed by exhaustive washing in a 10 kDa molecular weight cutoff Amicon centrifugal filter device and fluorescence was imaged. As shown in FIG. 5B, only protein containing the boronate amino acid showed fluorescence after labeling. FIG. 5B depicts the selective fluorescent labeling of Z-domain-K7(p-boronophenylalanine) (solid line) over Z-domain-K7Y (dotted line) with fluorescein-NHS-glucamine dye (FIG. 5A, Compound 3; R1=Fluorescein, R2=H). The yield of this protein labeling was determined to be approximately 61% based on the absorbance of the dye at 494 nm (ε₄₉₄˜65,000 cm⁻¹M⁻¹) and of the protein at 280 nm (ε₄₉₄˜24,750 cm⁻¹M⁻¹). Yields were lower than expected due to slight air oxidation of the boronic acid under these conditions.

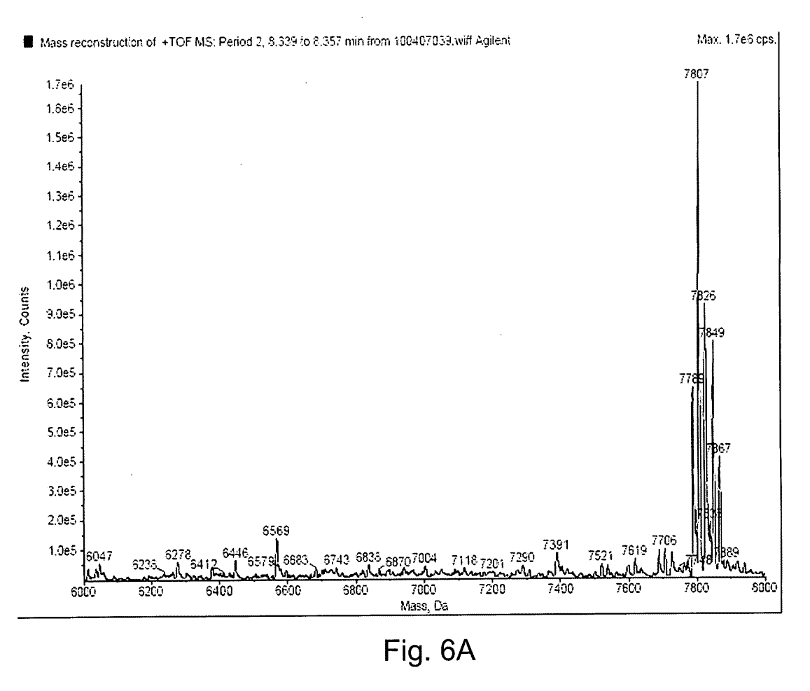

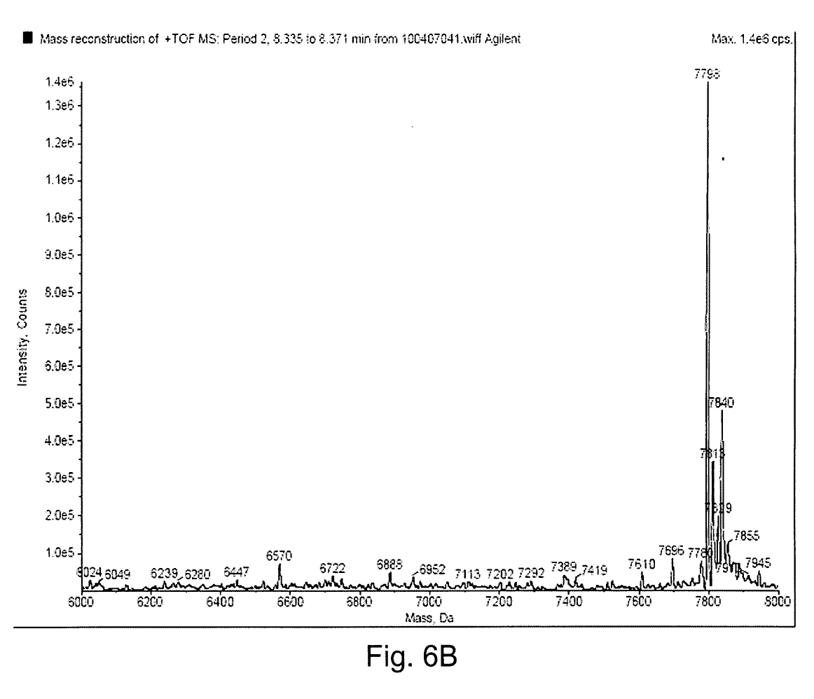

We next asked whether this chemistry could be used as a general strategy for the affinity purification of protein from cell lysates. XUS43594.00 resin 4, an immobilized glucamine affinity purification resin, (FIG. 5A, Compound 4; R1=polystyrene resin, R2═CH₃) from Dow Chemicals is an N-methylglucamine conjugated polystyrene resin designed for the removal of free borate from water supplies. To test whether a boronate amino acid would be sufficient for the affinity purification of a protein, 6×his tagged Z-domain with either p-boronophenylalanine or tyrosine at position 7 was expressed. After overnight expression in 2×YT media, clarified cell lysates in 50 mM CHES buffer, pH 8.5, were incubated with N-methylglucamine resin 4 (FIG. 5A, Compound 4) for 4 hrs at room temperature before being loaded onto a disposable polypropylene column and washed exhaustively with a high salt (1 M NaCl) wash buffer to remove any proteins that may stick to the resin through nonspecific interactions. The resin was then split and protein was eluted by the addition of excess sorbitol (1 M) or by oxidation of the boronate to tyrosine using excess hydrogen peroxide. FIG. 5C depicts the results of affinity purification of 6×his tagged Z-domain-K7(p-boronophenylalanine) using nickel-NTA resin or boronate affinity resin (Compound 4). Lane 1: protein markers; Lane 2: flow through of Ni-NTA purified protein; Lane 3: elution of Ni-NTA purified protein; Lane 4: flow through of boronate affinity resin; Lane 5: 200 mM hydrogen peroxide elution of boronate affinity resin; Lane 6: flow through of boronate affinity resin; Lane 7: 1 M sorbitol elution of boronate affinity resin. Z-domain is indicated in FIG. 5C by the arrow. As shown in FIG. 5C (lanes 5 and 7), Z-domain protein was isolated in high purity using either elution condition. Purification yields were comparable to that using Ni-NTA/His Tag purification (FIG. 5C, lane 3). Of 1 mg of the Z-domain-K7(p-boronophenylalanine) loaded onto 3 mL of the N-methylglucamine resin, 0.96 mg of protein was recovered in the sorbitol elution fraction corresponding to >95% protein recovery. Mass spectral analysis of the purified protein showed that protein eluted using high concentrations of competing sorbitol contained the unmodified boronate amino acid whereas hydrogen peroxide elution yielded exclusively protein containing tyrosine at that position (FIGS. 6A and 6B). FIG. 6A depicts the reconstructed ESI-TOF of a Z-domain glucamine resin sorbitol elution fraction. Peaks at 7807 (calc. 7807) and 7789 (calc. 7789) correspond to the boronate containing protein minus 1 or 2 waters, respectively, as described in FIG. 3A. FIG. 6B depicts the reconstructed ESI-TOF of Z-domain glucamine resin hydrogen peroxide elution fraction. Expected Z-domain-K7Y mass calc. 7798 (M+H); Obs. 7798. No protein was found in either elution fraction when tyrosine was used in place of the boronate amino acid (FIG. 5D, lanes 5 and 7). FIG. 5D depicts the results of affinity purification of Z-domain-K7Y using nickel-NTA resin or boronate affinity resin (Compound 5); Lane 1: protein markers; Lane 2: flow through of Ni-NTA purified protein; Lane 3: elution of Ni-NTA purified protein; Lane 4: flow through of boronate affinity resin; Lane 5: 200 mM hydrogen peroxide elution of boronate affinity resin; Lane 6: flow through of boronate affinity resin; Lane 7: 1 M sorbitol elution of boronate affinity resin. Z-domain is indicated in FIG. 5D by the arrow. It is significant to note that due to the ability of p-boronophenylalanine to be oxidized or reduced to tyrosine or phenylalanine, respectively, this methodology allows the purification of native protein sequences. In one step, a protein can be isolated based on the unique chemistry of the unnatural amino acid alone without the need for commonly used protein affinity tags such as 6×His or fusion proteins. Selective oxidation can be subsequently used to generate a tyrosine residue, yielding a native protein sequence with no modification remaining from the purification process.

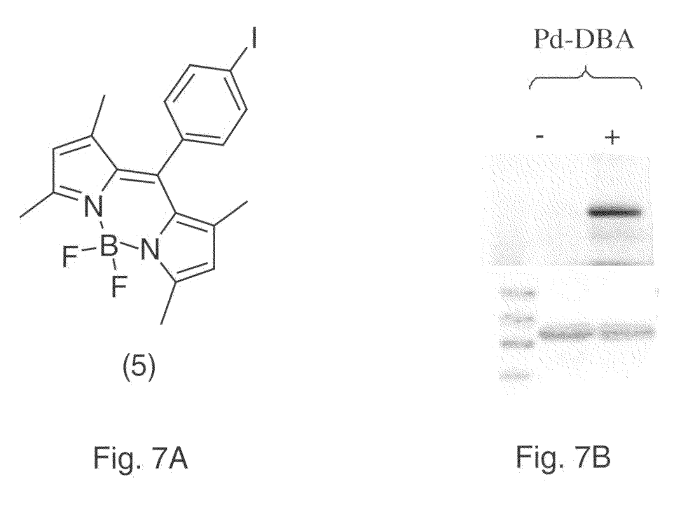

Finally, we explored the palladium mediated Suzuki coupling between boronate containing proteins and a fluorescent aryl-iodide reporter based on the BODIPY scaffold 5. FIG. 7A provides the structure of iodinated BODIPY reporter molecule Compound 5. The synthesis of BODIPY scaffold 5 was carried out by using a previously reported method with minor modifications (31). Suzuki couplings with proteins have already been reported with a synthetic W-domain peptide containing a p-iodophenylalanine unnatural amino acid and a fluorescent boronic acid compound in the presence of a water soluble palladium catalyst (Na₂PdCl₄) in Tris buffer (32). This procedure proved unsuccessful to couple BODIPY scaffold 5 to T4L-A82(p-boronophenylalanine). Previous experiments by our group have shown that poly-hydroxylated buffers such as Tris can inhibit certain reactions of boronic acids due to their ability to sequester boronates at high concentration. In addition Kodama, et al. have demonstrated the first example of a palladium catalyzed organometallic reaction on a biosynthetically incorporated p-iodophenylalanine amino acid. (33) To achieve Suzuki coupling of Compound 5 with T4L-A82(p-boronophenylalanine) a variety of basic buffered solutions and palladium catalysts were screened. In the end, coupling was achieved in moderate (ca. 30%) yield using the Pd⁰ dibenzylidene acetone (Pd-DBA) catalyst in 20 mM EPPS, pH 8.5, at 70° (FIG. 7B). FIG. 7B shows the results of Suzuki coupling of 50 μM T4L-A82(p-boronophenylalanine) to 1 mM reporter molecule (Compound 5) in 20 mM EPPS, pH 8.5, 70° C., for 12 hours in the absence (−) or presence (+) of 1 mM Pd-DBA. The top of FIG. 7B shows bodipy fluorescence, and the bottom shows Coomassie staining of the same gel. No coupling between the boronate protein and Compound 5 was observed in the absence of palladium catalyst. The high temperature needed to attain significant cross-coupling is most useful for proteins that are stable under these conditions. The development of more efficient water soluble palladium catalysts can improve this reactivity significantly (34, 35).

In summary, we have demonstrated the successful addition of the boronate functionality to the genetic code of E. coli in high yield and efficiency in response to the amber stop codon (TAG). The biosynthetic incorporation of this amino acid into proteins allows for selective chemistry on the protein surface including oxidation, reduction, Suzuki coupling reactions, as well as the formation of covalent boronic esters with polyhydroxylated compounds. We have also shown that this amino acid can be used in concert with a polyhydroxylated solid support to purify native protein sequences in a one step scarless affinity purification procedure. Furthermore, the ability of boronic acids to bind diols and reactive serine residues suggests that this technology could lead to the development of boronate-containing antibodies that specifically recognize and covalently bind various glycoproteins or proteases. It can also be possible to form intramolecular serine-boronate crosslinks in proteins to enhance stability. In addition, the unique chemistry of this functionality should allow for the in vivo labeling of boronate containing proteins with polyhydroxylated reporter molecules.

Materials

The unnatural amino acid p-boronophenylalanine was obtained from Aldrich. All chemicals used for synthesis are commercially available from Fisher Scientific or Aldrich. NHS-fluorescein was obtained from Pierce and the experimental resin XUS43594.00 (N-methylglucamine conjugated polystyrene) was purchased from Dow Chemicals. ESI mass spectrometry was performed on a single-Quad Agilent 1100 series LC-MS with either a tandem 1100 series ESI or 6100 series ESI-TOF.

Protein Expression

To express Z-domain-K7(p-boronophenylalanine), plasmid pLei-Z (which encodes the Z-domain-7TAG gene under a T7 IPTG-inducible promoter, the MjtRNA^{Tyr}_{CUA} under control of the lpp promoter, the low copy p15A origin, and a CAT gene for plasmid maintenance) was cotransformed with plasmid pBK-B(OH)₂PheRS (which contains the 1BG11 B(OH)₂PheRS under the control of the constitutive GlnS promoter, the high copy pMB1 origin of replication, and a kanamycin resistance gene for plasmid maintenance) into E. coli DH10B. Cells were grown in 2×YT media supplemented with 50 μg/mL kanamycin, 40 μg/mL chloramphenicol, and 1 mM p-boronophenylalanine to an OD₆₀₀ of 0.5 and induced by the addition of IPTG (1 mM final concentration). Cells were incubated at 37° C. for 16 hours and subsequently harvested by centrifugation. Z-domain protein was purified by Ni-affinity chromatography; 5 mg of protein was typically obtained per liter of cell culture. Expression of tyrosine controls was carried out by using the wild type Mj tyrosyl tRNA synthetase (plasmid pBK-JYRS) in place of the evolved B(OH)₂PheRS.

T4 lysozyme was expressed in a similar fashion to Z-domain with slight modifications. pLeiT4L82TAG is identical to pLeiZ but with a T4 lysozyme 82 amber mutant (no 6×His tag) in place of Z-domain. Cells were lysed by sonication in 50 mM MES, pH 6.5, 50 mM NaCl, and clarified lysates were initially purified by application to 10 mL of fast flow S-sepharose resin (GE-Healthcare). Protein was eluted in 2M NH₄OAc and dialyzed back into 50 mM MES, pH 6.5, 50 mM NaCl. Protein was purified to homogeneity on an ATKA P-900 FPLC using a monoS column (GE Healthcare) and a gradient salt ramp from 50 mM to 500 mM NaCl. Fractions containing purified T4 Lysozyme were pooled and dialyzed as appropriate for subsequent experiments.

Protein Oxidation and Reduction

To oxidize boronate containing proteins, 50-100 mM hydrogen peroxide was added to protein samples in 50 mM CHES buffer, pH 8.5. Oxidation was allowed to proceed for a minimum of 4 hrs at room temperature or alternatively heated to 50° C. for 30 minutes. Excess hydrogen peroxide was removed by desalting into water or a low salt CHES buffer, pH 8.5. Selective oxidation of boronate by oxone was performed on 50 μM protein in 10 mM sodium bicarbonate. Protein was cooled on ice for 1 hr before the addition of one equivalent of oxone. Oxidation was allowed to proceed for 5 minutes on ice before quenching by the addition of excess sodium bisulfite (10 mM). Protein was the exchanged into water for ESI. Reduction of boronate containing protein was performed using silver diammonia nitrate as previously described (36).

Synthesis of Fluorescent Reporter Molecules

Synthesis of glucamine conjugated fluorescein (FIG. 5A, Compound 4): 41 mg glucamine (0.23 mmol) was added to 5 ml 0.016 M aq. sodium bicarbonate. 0.1 g fluorescein-NHS ester (0.21 mmol) was dissolved in 1 ml DMF and then added to the glucamine bicarbonate solution dropwise. The reaction was allowed to proceed for two hours with stirring at room temperature. It was immediately loaded onto a C18 HPLC column and then eluted with a gradient of 25% to 35% acetonitrile in water over 4 column volumes. Fractions containing glucamine dye were lyophilized to dryness to give approximately >95% yield of purified product after HPLC. Compound 4 was analyzed by LC-MS: calcd M for C₂₇H₂₆NO₁₁, 540.1; found, 540.1. ¹H NMR (500 MHz, CD₃OD) δ 3.52-3.62 (m, 1H), δ 3.62-3.80 (m, 3H), δ 3.80-3.91 (m, 2H), 4.00-4.15 (m, 2H), 6.77 (d, J=8.0 Hz, 2H), 6.91 (d, J=8.0 Hz, 2H), 6.93 (s, 2H), 7.41 (d, J=8.0 Hz, 1H), 8.27 (d, J=8.0 Hz, 1H), 8.60 (s, 1H).

Synthesis of iodinated BODIPY reporter molecule (FIG. 7A, Compound 5) (37): the reaction mixture of 2,4-dimethyl-1H-pyrrole (82 mg, 0.86 mmol) and 4-iodo benzaldehyde (100 mg, 0.43 mmol) in anhydrous CH₂Cl₂ (40 mL) was treated with one drop of trifluoroacetic acid. The reaction was stirred at room temperature overnight. A solution of 2,3-dichloro-5,6-dicyano-1,4-benzoquinone (100 mg, 0.43 mmol) in anhydrous CH₂Cl₂ (10 mL) was added by syringe to the solution and stirring was continued for an additional 4 hrs. After addition of triethylamine (2 mL), BF₃OEt₂ (2 mL) was slowly added over 10 min at 0° C. The reaction mixture was stirred at room temperature for 4 hrs. After completion, the reaction mixture was extracted with CH₂Cl₂ and sat. NaHCO₃. The organic layer was dried over anhydrous sodium sulfate. The crude Compound 2 was purified by column chromatography (hexane:CH₂Cl₂=110:1) to afford an orange color solid (10 mg, 0.02 mmol, yield=4.7%). Compound 5 was analyzed by LC-MS: calculated M⁺ for C₁₉H₁₈BF₂IN₂, 450.1; found, 450.1. NMR analysis was consistent with the previously reported spectra (37).

Protein Labeling

Protein labeling with the glucamine-fluorescein reporter (FIG. 5A, Compound 4) was achieved by incubating 50 μM of boronate containing protein with 10 equivalents of Compound 4 in 50 mM CHES buffer, pH 8.5, 150 mM NaCl. Protein labeling was allowed to proceed for a minimum of 1 hour at room temperature. Excess dye was removed by filtration through a 10 kDa cutoff Amicon centrifugal filtration device (Millipore) followed by exhaustive washing with the 50 mM CHES labeling buffer. Labeling yields were determined by measuring the absorbance at 280 nm and 494 nm on a UVIKON UV spectrometer. The extinction coefficient used for the fluorescein dye at 494 was ˜65,000 cm⁻¹M⁻¹. An extinction coefficient of 24,750 for T4 lysozyme K82(p-boronophenylalanine) at 280 nm was used to determine protein concentration after subtracting off 30% the absorbance at 494 nm to account for fluorescein absorbance at 280 nm as determined by the spectrum of free dye in the same buffered solution.

Suzuki coupling between T4 lysozyme K82(p-boronophenylalanine) and Compound 5 was performed in 20 mM EPPS buffer, pH 8.5. 50 μM protein was mixed with 1 mM Compound 5 in buffer to give a 45 μL solution. 5 μL of a 10 mM suspension of Pd-DBA was then added to give a final concentration of 1 mM Pd catalyst. Reactions were incubated at 70° C. for 12 hours after which any particulate matter was removed by centrifugation and decanting. 20 μL of the crude reaction product was then loaded onto a 4-10% Tris-Glycine gel (Invitrogen) and separated by SDS-PAGE. Fluorescence was imaged on a Storm420 PhosphorImager using the blue fluorescence mode.

Boronate Affinity Protein Purifications

To purify proteins using the N-methylglucamine resin, proteins were expressed as described above. Cells were harvested by centrifugation and resuspended in boronate binding buffer (50 mM CHES buffer, pH 8.5, 150 mM NaCl) and lysed by incubation with 1 mg/mL hen egg white lysozyme for 1 hour followed by sonication. N-methylglucamine resin was added to clarified lysates and incubated at room temperature for 4 hours after which resin was loaded onto a 5 mL polypropylene column (Qiagen). After collection of the flow through, the resin was washed exhaustively with binding buffer followed by a high salt (50 mM CHES buffer, pH 8.5, 1 M NaCl) wash buffer. At this point, resin was split between multiple columns and the protein was eluted using either excess sorbitol (binding buffer+1M sorbitol) or hydrogen peroxide (binding buffer+100 mM H₂O₂ and incubation for 4 hours at room temperature). Protein was exchanged into water using a PD10 desalting column (GE healthcare), concentrated, and analyzed by SDS-PAGE.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

### REFERENCES

1. N. Miyaura and A. Suzuki, “Palladium-Catalyzed Cross-Coupling Reactions of Organoboron Compounds.” Chemical Reviews 1995, 95, 2457.
2. A. Suzuki, “Recent advances in the cross-coupling reactions of organoboron derivatives with organic electrophiles, 1995-1998.” Journal of Organometallic Chemistry 1999, 576, 147.
3. D. M. T. Chan, K. L. Monaco, R. H. Li, D. Bonne, C. G. Clark, and P. Y. S. Lam, “Copper promoted C—N and C—O bond cross-coupling with phenyl and pyridylboronates.” Tetrahedron Letters 2003, 44, 3863.
4. S. W. Huang, B. Wang, Z. X. Shan, D. J. Zhao, “Asymmetric reduction of acetophenone with borane catalyzed by chiral oxazaborolidinone derived from L-a-amino acids.” Synthetic Communications 2000, 30, 2423.
5. K. Ishihara, H. Yamamoto, “Arylboron Compounds as Acid Catalysts in Organic Synthetic Transformations.” European Journal of Organic Chemistry 1999, 527.
6. J. P. Lorand, J. O. Edwards, “Polyol Complexes and Structure of the Benzeneboronate Ion.” Journal of Organic Chemistry 1959, 24, 769.
7. G. Springsteen, C. E. Ballard, S. Gao, W. Wang, B. H. Wang, “The Development of Photometric Sensors for Boronic Acids.” Bioorganic Chemistry 2001, 29, 259.
8. L. K. Mohler, A. W. Czarnik, “Alpha-Amino acid Chelative complexation by an Arylboronic Acid.” Journal of the American Chemical Society 1994, 116, 2233.
9. L. K. Mohler, A. W. Czarnik, “Alpha-Amino-Acid Chelative Complexation by an Arylboronic Acid.” Journal of the American Chemical Society 1993, 115, 7037.
10. A. N. Cammidge, K. V. L. Crépy, “Synthesis of chiral binaphthalenes using the asymmetric Suzuki reaction.” Tetrahedron 2004, 60, 4377.
11. L. Lamandé, D. Boyer, A. Munoz, “Structure et acidite de composes a atome de bore et de phosphore hypercoordonnes.” Journal of Organometallic Chemistry 1980, 329, 1.
12. T. D. James, K. Sandanayake, S. Shinkai, “Saccharide Sensing with Molecular Receptors Based on Boronic Acid.” Angewandte Chemie-International Edition in English 1996, 35, 1910.
13. T. D. James, K. Sandanayake, S. Shinkai, “Chiral discrimination of monosaccharides using a fluorescent molecular sensor.” Nature 1995, 374, 345.
14. W. Wang, X. M. Gao, B. H. Wang, “Boronic Acid-Based Sensors.” Current Organic Chemistry 2002, 6, 1285.
15. J. Adams, M. Behnke, S. W. Chen, A. A. Cruickshank, L. R. Dick, L. Grenier, J. M. Klunder, Y. T. Ma, L. Plamondon, R. L. Stein, “Potent and selective inhibitors of the proteasome: Dipeptyidyl boronic acids.” Bioorganic & Medicinal Chemistry Letters 1998, 8, 333.
16. G. S. Weston, J. Blazquez, F. Baquero, B. K. Shoichet, “Structure-Based Enhancement of Boronic Acid Inhibitors of AmpC b-Lactamase.” Journal of Medicinal Chemistry 1998, 41, 4577.
17. W. Q. Yang, X. M. Gao, B. H. Wang, “Boronic acid compounds as potential pharmaceutical agents.” Medicinal Research Reviews 2003, 23, 346.
18. D. A. Matthews, R. A. Alden, J. J. Birktoft, S. T. Freer, J. Kraut, “X-ray crystallographic study of boronic acid adducts with subtilisin BPN′ (Novo). A model for the catalytic transition state.” Journal of Biological Chemistry 1975, 250, 7120.
19. Y. Kinashi, S. Masunaga, K. Ono, “Mutagenic effect of borocaptate sodium and boronophenylalanine in neutron capture therapy.” International Journal of Radiation Oncology Biology Physics 2002, 54, 562.
20. L. Wang, A. Brock, B. Herberich, P. G. Schultz, “Expanding the Genetic Code of Escherichia coli.” Science 2001, 292, 498.
21. J. W. Chin, T. A. Cropp, J. C. Anderson, M. Mukherji, Z. W. Zhang, P. G. Schultz, “An Expanded Eukaryotic Genetic Code.” Science 2003, 301, 964.
22. W. S. Liu, A. Brock, S. Chen, S. B. Chen, P. G. Schultz, “Genetic incorporation of unnatural amino acids into proteins in mammalian cells.” Nature Methods 2007, 4, 239.
23. L. Wang, T. J. Magliery, D. R. Liu, P. G. Schultz, “A New Functional Suppressor tRNA/Aminoacyl-tRNA Synthetase Pair for the in Vivo Incorporation of Unnatural Amino Acids into Proteins.” Journal of the American Chemical Society 2000, 122, 5010.
24. L. Wang, P. G. Schultz, “Expanding the genetic code.” Chemical Communications 2002, 1.
25. J. M. Xie, W. S. Liu, P. G. Schultz, “A Genetically Encoded Bidentate, Metal-Binding Amino Acid.” Angewandte Chemie-International Edition 2007, 46, 9239.
26. T. Kobayashi, K. Sakamoto, T. Takimura, R. Sekine, K. Vincent, K. Kamata, S, Nishimura, S. Yokoyama, “Structural basis of normatural amino acid recognition by an engineered aminoacyl-tRNA synthetase for genetic code expansion.” Proceedings of the National Academy of Sciences of the United States of America 2005, 102, 1366.
27. D. Y. Zheng, J. M. Aramini, G. T. Montelione, “Validation of helical tilt angles in the solution NMR structure of the Z domain of Staphylococcal protein A by combined analysis of residual dipolar coupling and NOE data.” Protein Science 2004, 13, 549.
28. D. S. Kemp, D. C. Roberts, “New protective groups for peptide synthesis—II the Dobz group boron-derived affinity protection with the p-dihydroxyborylbenzyloxycarbonylamino function.” Tetrahedron Letters 1975, 16, 4629.
29. K. S. Webb, D. Levy, “A facile oxidation of boronic acids and boronic esters.” Tetrahedron Letters 1995, 36, 5117.
30. G. Springsteen, B. H. Wang, “A detailed examination of boronic acid-diol complexation.” Tetrahedron 2002, 58, 5291.
31. C. Tahtaoui, C. Thomas, F. Rohmer, P. Klotz, G. Duportail, Y. Mely, D. Bonnet, M. Hibert, “Convenient Method To Access New 4,4-Dialkoxy- and 4,4-Diaryloxy-diaza-s-indacene Dyes: Synthesis and Spectroscopic Evaluation.” Journal of Organic Chemistry 2007, 72, 269.
32. A. Ojida, H. Tsutsumi, N. Kasagi, I. Hamachi, “Suzuki coupling for protein modification.” Tetrahedron Letters 2005, 46, 3301.
33. K. Kodama, S. Fukuzawa, H. Nakayama, T. Kigawa, K. Sakamoto, T. Yabuki, N. Matsuda, M. Shirouzu, K. Takio, K. Tachibana, S. Yokoyama, “Regioselective Carbon-Carbon Bond Formation in Proteins with Palladium Catalysis; New Protein Chemistry by Organometallic Chemistry.” ChemBioChem 2006, 7, 134.
34. D. Badone, M. Baroni, R. Cardamone, A. Ielmini, U. Guzzi, “Highly Efficient Palladium-Catalyzed Boronic Acid Coupling Reactions in Water: Scope and Limitations.” Journal of Organic Chemistry 1997, 62, 7170.
35. K. H. Shaughnessy, R. S. Booth, Abstracts of Papers of the American Chemical Society 2004, 227, U1534.
36. D. S. Kemp, David C. Roberts, “New protective groups for peptide synthesis-II the Dobz group boron-derived affinity protection with the p-dihydroxyborylbenzyloxycarbonylamino function Tett. Lett. 1975, 16, 4629.
37. Tahtaoui, C; Thomas, C; Rohmer, F; Klotz, P; Duportail, G; Me'ly, Y; Bonnet, D; Hibert, M., “Convenient Method To Access New 4,4-Dialkoxy- and 4,4-Diaryloxy-diaza-s-indacene Dyes: Synthesis and Spectroscopic Evaluation.” J. Org. Chem. 2007, 72, 269-272.

1. A composition comprising an aminoacyl tRNA synthetase that selectively recognizes a boronic amino acid.

2. The composition of claim 1, wherein the aminoacyl tRNA synthetase selectively recognizes an aliphatic, aryl or heterocycle substituted boronic acid, a p-boronophenylalanine, an o-boronophenylalanine, or an m-boronophenylalanine.

3. The composition of claim 1, wherein the synthetase is homologous to a wild-type tyrosyl tRNA synthetase from Methanococcus jannaschii.

4. The composition of claim 3, wherein the synthetase comprises: a Ser or Gly residue at position 32, an alanine at position 65, a His or Met residue at position 70, a Ser or Ala residue at position 158, a glutamine at position 162 or a combination thereof, wherein amino acid position numbering corresponds to amino acid position numbering of the wild-type tyrosyl tRNA synthetase.

5. The composition of claim 4, wherein the synthetase is encoded by: 1BF6 (SEQ ID NO: 2), 1BF9 (SEQ ID NO: 3), 1BE3 (SEQ ID NO: 4), 1BF10 (SEQ ID NO: 5), 1BF12 (SEQ ID NO: 6), 1BG10 (SEQ ID NO: 7), or 1BG11 (SEQ ID NO: 8).

6. The composition of claim 1, comprising a cell, which cell expresses the aminoacyl tRNA synthetase, wherein the synthetase is orthogonal to the cell, and wherein the cell further expresses a cognate orthogonal tRNA (OtRNA) that is selectively charged by the synthetase with the boronic amino acid.

7. The composition of claim 6, wherein the OtRNA comprises a sequence as shown in the sequence listing.

8. The composition of claim 6, wherein the cell is a bacterial or eukaryotic cell.

9. The composition of claim 8, wherein the cell is an E. coli, yeast or mammalian cell.

10. The composition of claim 6, the cell encoding a target nucleic acid, wherein the OtRNA selectively recognizes a selector codon in the target nucleic acid, wherein the cell specifically incorporates a boronic amino acid residue into a target polypeptide in response to the selector codon.

11. The composition of claim 10, wherein the selector codon is a stop codon, a rare codon, a nonsense codon, or a 4 or more base codon.

12. The composition of claim 10, wherein the target polypeptide comprising the boronic amino acid residue is a substrate for a labeling reaction, a substrate for probe addition, a substrate for an oxidation reaction, a substrate for a reduction reaction, a substrate for an esterification reaction, a substrate for a saccharide addition reaction, a substrate for a PEG addition reaction, a substrate for polyol addition, a substrate for a Suzuki cross-coupling reaction, a substrate for a transition metal catalyzed reaction, a palladium catalyzed reaction, a substrate for a copper catalyzed heteroatom alkylation reaction, a substrate for an asymmetric reduction, or a substrate for a Diels-Alder reaction, wherein the respective reaction selectively acts on the boronic amino acid residue.

13. The composition of claim 10, wherein the target polypeptide is a therapeutic protein, a cytokine, a growth factor, an immunogen, an enzyme, a cell receptor ligand, a modulator of a serine protease, an inhibitor of a serine protease, a modulator of a glycosylated macromolecule, an inhibitor of a glycosylated macromolecule, a saccharide binding protein, an oligosaccharide binding protein, an antibody, an antibody fragment, a therapeutic antibody, an antibody or antibody fragment that specifically binds to a glycoprotein, an antibody that specifically binds to a serine protease, an antibody that specifically binds to a serum protease, a phage display protein, or a cancer cell ligand.

14. A method of incorporating a boronic amino acid into a target polypeptide, the method comprising:
providing a translation system comprising an orthogonal aminoacyl tRNA synthetase (ORS) selective for the boronic amino acid, the translation system further comprising a cognate orthogonal tRNA (OtRNA) specific for a selector codon, and a target nucleic acid encoding the target polypeptide and comprising the selector codon; and,
permitting the translation system to incorporate a boronic amino acid residue into the target polypeptide during translation of the target nucleic acid into the target polypeptide.

15-40. (canceled)

41. A method of producing a protein, the method comprising: site-specifically encoding a boronic amino acid residue into a mutant protein; and, selectively converting the boronic amino acid residue into a natural amino acid residue, thereby producing the protein.

42-45. (canceled)

46. A composition comprising a solid phase matrix covalently bound to a polypeptide through a boronic amino acid residue.

47-48. (canceled)

49. A composition comprising a purified population of polypeptide molecules that each comprise a borono amino acid at a selected site.

50-56. (canceled)


SEQUENCE LISTING (19 sequences)

SEQ ID NO: 1 - DNA, 921 bp, Methanococcus jannaschii
atggacgaatttgaaatgataaagagaaacacatctgaaattatcagcgaggaagagttaagagaggttttaaaaaaagatgaaaaatctgcttacataggttttgaaccaagtggtaaaatacatttagggcattatctccaaataaaaaagatgattgatttacaaaatgctggatttgatataattatattgttggctgatttacacgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaatgatattcattatttaggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 2 - DNA, 921 bp, Artificial Sequence
  Aminoacyl tRNA synthetase derived from Methanococcus jannaschii tyrosyl-tRNA synthetase.  Clone 1BF6.
atggacgaatttgaaatgataaagagaaacacatctgaaattatcagcgaggaagagttaagagaggttttaaaaaaagatgaaaaatctgcttcgataggttttgaaccaagtggtaaaatacatttagggcattatctccaaataaaaaagatgattgatttacaaaatgctggatttgatataattatagcgttggctgatttacatgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaattctattcattatgagggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 3 - DNA, 921 bp, Artificial Sequence
  Aminoacyl tRNA synthetase derived from Methanococcus jannaschii tyrosyl-tRNA synthetase.  Clone 1BF9.
atggacgaatttgaaatgataaagagaaacacatctgaaattatcagcgaggaagagttaagagaggttttaaaaaaagatgaaaaatctgcttcgataggttttgaaccaagtggtaaaatacatttagggcattatctccaaataaaaaagatgattgatttacaaaatgctggatttgatataattatagctttggctgatttacatgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaattctattcattatgagggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 4 - DNA, 921 bp, Artificial Sequence
  Aminoacyl tRNA synthetase derived from Methanococcus jannaschii tyrosyl-tRNA synthetase.  Clone 1BE3.
atggacgaatttgaaatgataaagagaaacacatctgaaattatcagcgaggaagagttaagagaggttttaaaaaaagatgaaaaatctgctggtataggttttgaaccaagtggtaaaatacatttagggcattatctccaaataaaaaagatgattgatttacaaaatgatggatttgatataattatagctttggctgatttacatgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaatgcgattcattatgagggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 5 - DNA, 921 bp, Artificial Sequence
  Aminoacyl tRNA synthetase derived from Methanococcus jannaschii tyrosyl-tRNA synthetase.  Clone 1BF10.
atggacgaatttgaaatgataaagagaaacacatctgaaattatcagcgaggaagagttaagagaggttttaaaaaaagatgaaaaatctgcttctataggttttgaaccaagtggtaaaatacatttagggcattatctccaaataaaaaagatgattgatttacaaaatgctggatttgatataattatagcgttggctgatttaatggcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaattctattcattatgagggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 6 - DNA, 921 bp, Artificial Sequence; identical to SEQ ID NO: 5
  Aminoacyl tRNA synthetase derived from Methanococcus jannaschii tyrosyl-tRNA synthetase.  Clone 1BF12.

SEQ ID NO: 7 - DNA, 921 bp, Artificial Sequence
  Aminoacyl tRNA synthetase derived from Methanococcus jannaschii tyrosyl-tRNA synthetase.  Clone 1BG10.
atggacgaatttgaaatgataaagagaaacacatctgaaattatcagcgaggaagagttaagagaggttttaaaaaaagatgaaaaatctgcttcgataggttttgaaccaagtggtaaaatacatttagggcattatctccaaataaaaaagatgattgatttacaaaatgctggatttgatataattatagctttggctgatttaatggcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaattctattcattatgagggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 8 - DNA, 921 bp, Artificial Sequence; identical to SEQ ID NO: 5
  Aminoacyl tRNA synthetase derived from Methanococcus jannaschii tyrosyl-tRNA synthetase. Clone 1BG11.

SEQ ID NO: 9 - protein, 306 aa, Methanococcus jannaschii
MDEFEMIKRNTSEIISEEELREVLKKDEKSAYIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNDIHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 10 - protein, 306 aa, Artificial Sequence
  Aminoacyl tRNA synthetase derived from Methanococcus jannaschii tyrosyl-tRNA synthetase.  Clone 1BF6.
MDEFEMIKRNTSEIISEEELREVLKKDEKSASIGFEPSGKIHLGHYLQIKKMIDLQNAGFDIIIALADLHAYLNQKGELDEIRKIGDYNKKVFEAMGLKAKYVYGSEFQLDKDYTLNVYRLALKTTLKRARRSMELIAREDENPKVAEVIYPIMQVNSIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 11 - protein, 306 aa, Artificial Sequence; identical to SEQ ID NO: 10
  Aminoacyl tRNA synthetase derived from Methanococcus jannaschii tyrosyl-tRNA synthetase.  Clone 1BF9.

SEQ ID NO: 12 - protein, 306 aa, Artificial Sequence
  Aminoacyl tRNA synthetase derived from Methanococcus jannaschii tyrosyl-tRNA synthetase.  Clone 1BE3.
MDEFEMIKRNTSEIISEEELREVLKKDEKSAGIGFEPSGKIHLGHYLQIKKMIDLQNDGFDIIIALADLHAYLNQKGELDEIRKIGDYNKKVFEAMGLKAKYVYGSEFQLDKDYTLNVYRLALKTTLKRARRSMELIAREDENPKVAEVIYPIMQVNAIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 13 - protein, 306 aa, Artificial Sequence
  Aminoacyl tRNA synthetase derived from Methanococcus jannaschii tyrosyl-tRNA synthetase.  Clone 1BF10.
MDEFEMIKRNTSEIISEEELREVLKKDEKSASIGFEPSGKIHLGHYLQIKKMIDLQNAGFDIIIALADLMAYLNQKGELDEIRKIGDYNKKVFEAMGLKAKYVYGSEFQLDKDYTLNVYRLALKTTLKRARRSMELIAREDENPKVAEVIYPIMQVNSIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 14 - protein, 306 aa, Artificial Sequence; identical to SEQ ID NO: 13
  Aminoacyl tRNA synthetase derived from Methanococcus jannaschii tyrosyl-tRNA synthetase.  Clone 1BF12.

SEQ ID NO: 15 - protein, 306 aa, Artificial Sequence; identical to SEQ ID NO: 13
  Aminoacyl tRNA synthetase derived from Methanococcus jannaschii tyrosyl-tRNA synthetase.  Clone 1BG10.

SEQ ID NO: 16 - protein, 306 aa, Artificial Sequence; identical to SEQ ID NO: 13
  Aminoacyl tRNA synthetase derived from Methanococcus jannaschii tyrosyl-tRNA synthetase.  Clone 1BG11.

SEQ ID NO: 17 - DNA, 77 bp, Methanococcus jannaschii
ccggcggtagttcagcagggcagaacggcggactctaaatccgcatggcgctggttcaaatccggcccgccggacca

SEQ ID NO: 18 - protein, 69 aa, Artificial Sequence
  Z domain protein comprising the unnatural amino acid p-boronophenylalanine at amino acid position 7.
MTSVDNXINKEQQNAFYEILHLPNLNEEQRDAFIQSLKDDPSQSANLLAEAKKLNDAQAPKGSHHHHHH

SEQ ID NO: 19 - protein, 164 aa, Artificial Sequence
  T4 lysozyme protein comprising the unnatural amino acid p-boronophenylalanine at amino acid position 82.
MNIFEMLRIDEGLRLKIYKDTEGYYTIGIGHLLTKSPSLNAAKSELDKAIGRNTNGVITKDEAEKLFNQDVDAAVRGILRNXKLKPVYDSLDAVRRAALINMVFQMGETGVAGFTNSLRMLQQKRWDEAAVNLAKSRWYNQTPNRAKRVITTFRTGTWDAYKNL